(19) United States
(12) Patent Application Publication
(10) Pub. No.: US 2024/0318072 A1
(43) Pub. Date: Sep. 26, 2024

(54) PHOTOACTIVE MATERIAL

(71) Applicant: Sumitomo Chemical Co., Ltd, Tokyo (JP)

(72) Inventors: Florence Bourcet, Godmanchester (GB); Michal Maciejczyk, Godmanchester (GB)

(73) Assignee: Sumitomo Chemical Co., Ltd, Tokyo (JP)

(21) Appl. No.: 18/268,112

(22) PCT Filed: Dec. 17, 2021
(86) PCT No.: PCT/EP2021/086628

§ 371 Date: Jun. 16, 2023

(30) Foreign Application Priority Data
Dec. 18, 2020 (GB) 2020185.1

(51) Int. Cl. C09K 11/06; H10K 30/30; H10K 71/12; H10K 85/60

(52) U.S. Cl. CPC C09K 11/06; H10K 30/30; H10K 71/12; H10K 85/654; C09K 2211/1029

(57) ABSTRACT

A material comprising an electron-accepting unit of formula (I). According to some embodiments, the present disclosure provides a material comprising an electron-accepting unit of formula (I) wherein Ar is a substituted or unsubstituted benzene or 6-membered heteroaromatic ring containing N and C ring atoms; Ar¹ is a substituted or unsubstituted 5- or 6-membered heteroaromatic ring containing N and C ring atoms; Ar² is a substituted or unsubstituted 5- or 6-membered heteroaromatic ring or is absent; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a carbon atom of Ar³ and, where present, Ar⁴ with the proviso that at least one X group is an electron-withdrawing group and wherein the material further comprises a conjugated electron-donating unit. The material may be a polymer comprising repeat units of formula (I). The material may be a non-polymeric compound. An organic photodetector may contain a bulk heterojunction layer containing an electron acceptor or an electron donor wherein at least one of the electron acceptor and electron donor contains a unit of formula (I).

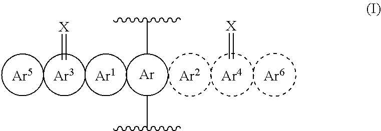

### BACKGROUND

Embodiments of the present disclosure relate to photoactive materials and more specifically, but not by way of limitation, to photoactive materials containing an electron-accepting unit and an electron-donating unit, the materials being suitable for use as an electron-donating material or an electron-accepting material in a photoresponsive device.

Q. Zhang et al, “Novel carbazole-based donor-isoindolo[2,1-a]benzimidazol-11-one acceptor polymers for ternary flash memory and light-emission”, RSC Advances (2019), 9(47), p 27665-27673, discloses compounds based on 9-(9-heptadecanyl)-9H-carbazole and isoindolo[2,1-a]benzimidazol-11-one with fluorine substituents on the acceptor unit.

Q. Zhang et al, “Novel Conjugated Side Chain Fluorinated Polymers Based on Fluorene for Light-Emitting and Ternary Flash Memory Devices”, ChemPubSoc Europe, (2019), 8, p 1267-1275, discloses conjugated polymers based on 9,9′-dioctylfluorene and isoindolo[2,1-a]benzimidazol-11-one units with different fluorine substitution.

H. Zhang et al, “Ternary Memory Devices Based on Bipolar Copolymers with Naphthalene Benzimidazole Acceptors and Fluorene/Carbazole Donors”, discloses donor-acceptor-type bipolar conjugated copolymers.

H. Chen et al, “Low Band Gap Donor-Acceptor Conjugated Polymers with Indanone-Condensed Thiadiazolo[3,4-g]quinoxaline Acceptors”, Macromolecules, (2019), 52(16), p 6149-6159, discloses compounds of formula (II) and (III) is directed to polymers based on 2,5-bis(3-(2-decyltetradecyl)thiophen-2-yl)thieno[3,2-b]-thiophene units as the electron donor and thiadiazolo[3,4-g]quinoxaline acceptor units.

J. Chen et al, “D-A Conjugated Polymers based on Tetracyclic Acceptor Units: Synthesis and Application in Organic Solar Cells”, Macromol. Chem. Phys., (2013), 214(18), p 2054-2060, discloses polymers based on indeno-pyrazine and indeno-quinoxaline units.

CN110128631A discloses super low band gap D-A conjugated polymers of formula (I) and (II), for FETs, NIR light detectors, NIR electrochromic devices and biological imaging applications.

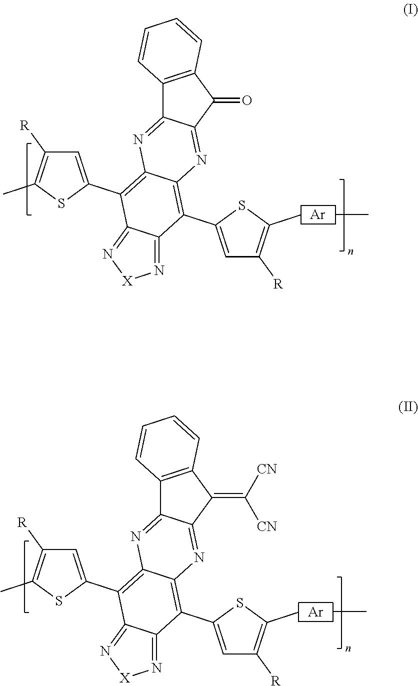

CN101376686A discloses narrow band gap, polymeric donor materials for bulk heterojunction solar cells based on carbazole, fluorene, phenyl and thiophene donor units.

### SUMMARY

According to some embodiments, the present disclosure provides a material comprising an electron-accepting unit of formula (I):

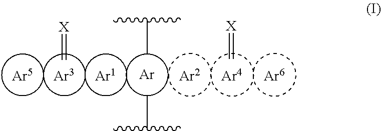

wherein Ar is a substituted or unsubstituted benzene or 6-membered heteroaromatic ring containing N and C ring atoms; Ar¹ is a substituted or unsubstituted 5- or 6-membered heteroaromatic ring containing N and C ring atoms; Ar² is a substituted or unsubstituted 5- or 6-membered heteroaromatic ring or is absent; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a carbon atom of Ar³ and, where present, Ar⁴ with the proviso that at least one X group is an electron withdrawing group, and wherein the material further comprises a conjugated electron-donating unit D of formula (II):

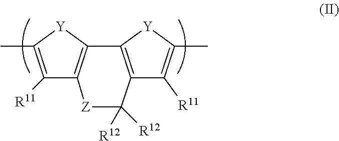

wherein:
Y in each occurrence is independently O, S or Se;
Z is O, S, C═O or NR³ wherein R³ is H or a substituent;
R¹¹ in each occurrence is independently H or a substituent; and
R¹² in each occurrence is independently a substituent.

It will be understood that the electron withdrawing group X of Ar³ and Ar⁴ may be attached by its double bond to any available C atom of Ar³ and Ar⁴.

Optionally, the unit of formula (1) is selected from formulae (I-1)-(I-31) according to claim 2.

Optionally, each electron-withdrawing group X is independently selected from O, S and NX⁷⁰ wherein X⁷⁰ is CN, COOR⁸⁰ or C₁ to C₂₀ alkyl chain where any non-terminal C can be replaced by O or S, substituted or unsubstituted 5- or 6-membered aromatic or heteroaromatic ring; and CX¹⁰X¹¹ wherein X¹⁰ and X¹¹ are each independently F, Cl, Br, CN, CF₃, or COOR⁸⁰, wherein R⁸⁰ is H or a substituent.

In some embodiments, the material is a non-polymeric compound. Optionally, the non-polymeric compound is selected from formulae (Ia)-(Id):

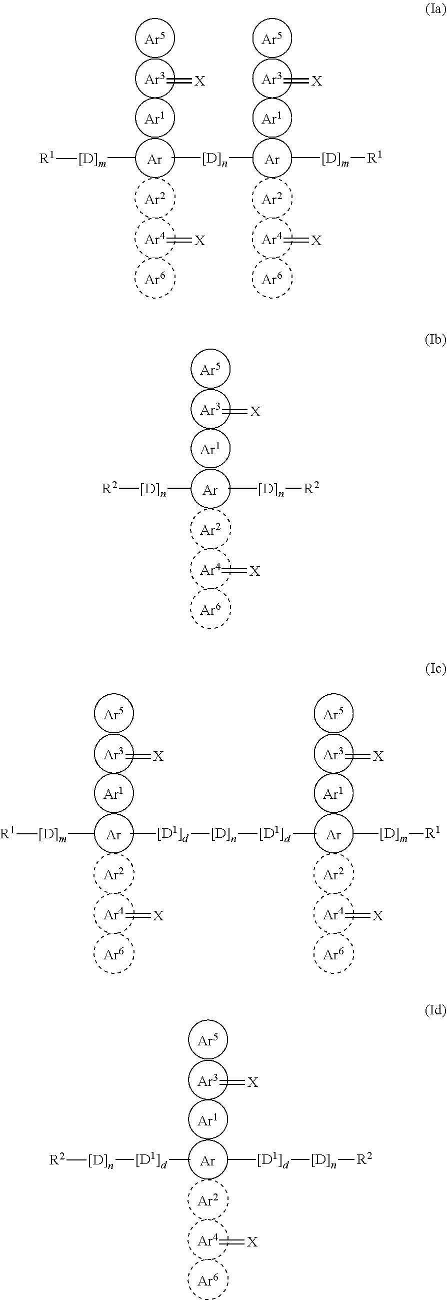

wherein n is at least 1; m is 0, 1, 2 or 3; d is 0, 1 or 2; D in each occurrence is independently a conjugated electron-donating unit D of formula (II); R¹ and R² independently in each occurrence is H or a substituent; and D¹ is a conjugated bridge unit which is different from D.

The conjugated bridge units D¹ as described herein are disposed between the electron-accepting unit of formula (I) and electron-donating unit D of formula (II), and preferably directly linked to the units of formula (I) and formula (II).

In some embodiments, the material is a polymer; the unit of formula (I) is an electron-accepting repeat unit of formula (I); and the conjugated electron-donating unit D is an electron-donating repeat unit of formula (II).

In some embodiments, the polymer further comprises an electron-donating repeat structure D¹ which is different from electron-donating unit D, and the polymer has a repeating structure of formula (Va):

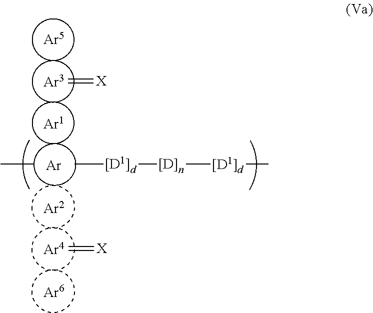

According to some embodiments, the present disclosure provides a composition comprising an electron donor and an electron acceptor wherein at least one of the electron donor and electron acceptor is a material comprising an electron-accepting unit of formula (I) and an electron-donating unit D of formula (II) as described herein.

In some embodiments, the electron acceptor of the composition is the material comprising an electron-accepting unit of formula (I) as described herein. Optionally according to these embodiments, the electron acceptor is a non-polymeric compound as described herein.

In some embodiments, the electron donor is the material comprising an electron-accepting unit of formula (I) as described herein. Optionally, the electron donor is a polymer as described herein.

According to some embodiments, the present disclosure provides an organic electronic device comprising an active layer comprising a material or composition as described herein.

Optionally, the organic electronic device is an organic photoresponsive device comprising a bulk heterojunction layer disposed between an anode and a cathode and wherein the bulk heterojunction layer comprises a composition as described herein.

Preferably, the organic photoresponsive device is an organic photodetector.

According to some embodiments, the present disclosure provides a photosensor comprising a light source and an organic photodetector as described herein, wherein the photosensor is configured to detect light emitted from a light source. Optionally, the light source emits light having a peak wavelength of >1250 nm and more preferably >1300 nm. Optionally, the light source emits light having a peak wavelength of no more than 1600 nm.

According to some embodiments, the present disclosure provides a formulation comprising a material or composition as described herein, dissolved or dispersed in one or more solvents.

According to some embodiments, the present disclosure provides a method of forming an organic electronic device as described herein, wherein formation of the active layer comprises deposition of a formulation as described herein onto a surface and evaporation of the one or more solvents.

### DESCRIPTION OF DRAWINGS

The disclosed technology and accompanying figures describe some implementations of the disclosed technology.

FIG. 1 illustrates an organic photoresponsive device according to some embodiments;

FIG. 2 shows toluene solution absorption spectra for a comparative electron-donating polymer and electron-donating polymers according to embodiments of the present disclosure;

FIG. 3 shows current densities vs. voltage for the organic photodetectors according to some embodiments which are not exposed to light;

FIG. 4 shows external quantum efficiencies vs. wavelength for organic photodetectors containing an electron donor polymer according to some embodiments and a comparative organic photodetector containing a comparative electron donor polymer;

FIG. 5 shows external quantum efficiencies vs. wavelength for organic photodetectors containing an electron donor polymer according to some embodiments of the present disclosure;

FIG. 6 shows external quantum efficiency vs. wavelength for an organic photodetector containing an electron donor polymer according to some embodiments of the present disclosure and a fullerene acceptor; and

FIG. 7 shows external quantum efficiency vs. wavelength for a comparative organic photodetector containing a comparative electron donor polymer and a fullerene acceptor.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers are may be present. References to a layer “on” another layer when used in this application means that the layers are in direct contact.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

A material as described herein may be provided in a bulk heterojunction layer of a photoresponsive device, preferably a photodetector, in which the bulk heterojunction layer is disposed between an anode and a cathode.

The bulk heterojunction layer comprises an electron donor material and an electron acceptor material wherein at least one of the electron donor material and the electron acceptor material comprises an electron-accepting group of Formula (I):

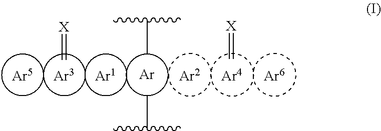

wherein Ar is a substituted or unsubstituted benzene or 6-membered heteroaromatic ring containing N and C atoms; Ar¹ is a substituted or unsubstituted 5- or 6-membered heteroaromatic ring containing N and C atoms; Ar² is a substituted or unsubstituted 5- or 6-membered heteroaromatic ring or is absent; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a carbon atom of Ar³ and, where present, Ar⁴ with the proviso that at least one X is an electron withdrawing group and wherein the material further comprises a conjugated electron-donating unit D of formula (II):

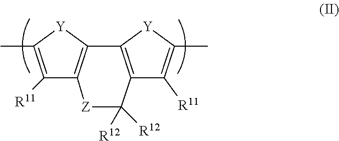

wherein:
Y in each occurrence is independently O, S or Se;
Z is O, S, C═O or NR³ wherein R³ is H or a substituent;
R¹¹ in each occurrence is independently H or a substituent; and
R¹² in each occurrence is independently a substituent.

It will be understood that the electron withdrawing group X of Ar³ and Ar⁴ may be attached by its double bond to any available C atom of Ar³ and Ar⁴.

Preferably, each R¹² is independently selected from the group consisting of:
linear, branched or cyclic C₁₋₂₀ alkyl wherein one or more non-adjacent, non-terminal C atoms may be replaced by O, S, NR⁸, CO or COO wherein R⁸ is a C₁₋₁₂ hydrocarbyl and one or more H atoms of the C₁₋₂₀ alkyl may be replaced with F; and
a group of formula -(Ak)u-(Ar⁷)v wherein Ak is a C₁₋₁₂ alkylene chain in which one or more non-adjacent C atoms may be replaced with O, S, CO or COO; u is 0 or 1; Ar⁷ in each occurrence is independently an aromatic or heteroaromatic group which is unsubstituted or substituted with one or more substituents; and v is at least 1.

Substituents of Ar⁷, if present, are preferably selected from F; Cl; NO₂; CN; and C₁₋₁₂ alkyl wherein one or more non-adjacent C atoms may be replaced with O, S, CO or COO Preferably, Ar⁷ is phenyl.

By “non-terminal” C atom of an alkyl group as used anywhere herein is meant a C atom of the alkyl other than the methyl C atom of a linear (n-alkyl) chain or the methyl C atoms of a branched alkyl chain.

Optionally, each R¹¹ is independently selected from H, F and a substituent as described with reference to R¹². Preferably, each R¹¹ is H.

Preferably, R³ is a C₁₋₂₀ hydrocarbyl group, optionally a C₁₋₂₀ alkyl; unsubstituted phenyl; or phenyl substituted with one or more C₁₋₁₂ alkyl groups.

Exemplary repeat units of formula (II) include, without limitation:

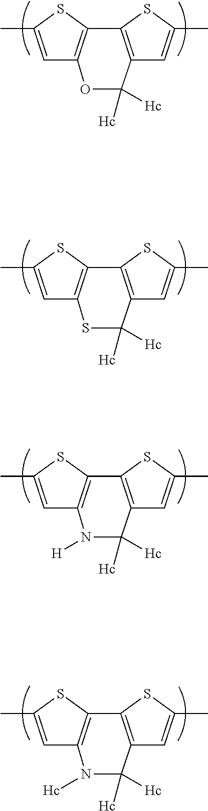

wherein Hc in each occurrence is independently a C₁₋₂₀ hydrocarbyl group, e.g. C₁₋₂₀ alkyl, unsubstituted aryl, or aryl substituted with one or more C₁₋₁₂ alkyl groups. The aryl group is preferably phenyl.

In some embodiments, no electron-donating groups D of formula (II) are directly linked to one another.

In some embodiments, the material comprises electron-donating groups that are linked directly to one another, e.g. to form a group of formula -(D)ₙ- or -(D)ₘ- wherein n or m is greater than 1, e.g. 2 or 3. In these embodiments, the groups D of formula (II) may in each occurrence be the same or different and may be linked in any orientation.

For example, in the case where n or m is 2 -(D)ₙ- or -(D)ₘ- may be selected from any of:

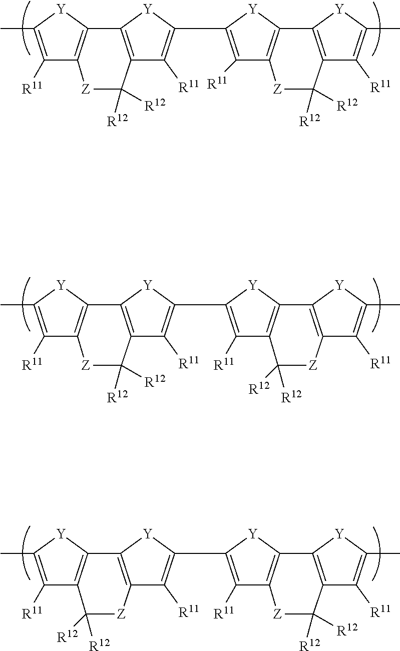

Carbon atoms of Ar¹, Ar², Ar³, Ar⁴, Ar⁵ and Ar⁶ which are not fused to another ring or substituted with X carry a group R⁶¹ wherein R⁶¹ in each occurrence is independently H or a substituent. Substituents R⁶¹ are preferably selected from the group consisting of:
F;
Cl;
CN;
NO₂;
linear, branched or cyclic C₁₋₂₀ alkyl wherein one or more non-adjacent, non-terminal C atoms may be replaced by O, S, NR⁷, CO or COO wherein R⁷ is a C₁₋₁₂ hydrocarbyl and one or more H atoms of the C₁₋₂₀ alkyl may be replaced with F; and
a group of formula (Ak)u-(Ar⁷)v wherein Ak is a C₁₋₁₂ alkylene chain in which one or more non-adjacent C atoms may be replaced with O, S, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₁₂ alkyl wherein one or more non-adjacent C atoms may be replaced with O, S, CO or COO Preferably, Ar⁷ is phenyl.

More preferred substituents R⁶¹ are F; Cl; C1-20 alkyl wherein one or more H atoms may be replaced with F; and phenyl which is unsubstituted or substituted with one or more substituents selected from F and C₁₋₁₂ alkyl wherein one or more H atoms of the alkyl may be replaced with F.

It will be understood that the possibility of substituting Ar¹, Ar², Ar⁵ and Ar⁶ will be dependent on the structure of formula (I) and the availability of substitution positions. For example, if Ar² is present and is a 6-membered aromatic or heteroaromatic ring containing less than four heteroatoms in the ring, then substitution may be present; if Ar² is a 5-membered heteroaromatic ring, containing less than three heteroatoms in the ring, then substitution may be present; if Ar⁵ is a monocyclic or polycyclic group containing a least one aromatic ring, then substitution may be present.

Preferably, the unit of formula (I) is selected from formulae (I-1)-(I-31):

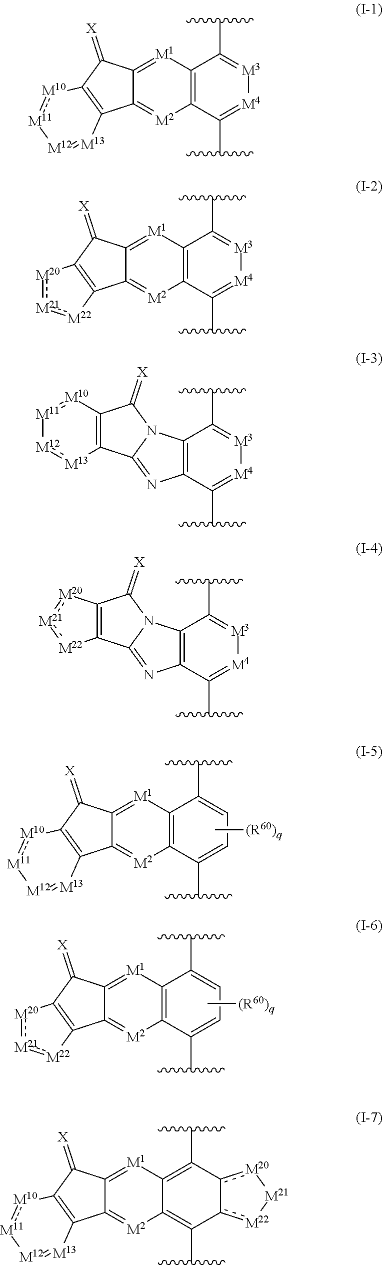

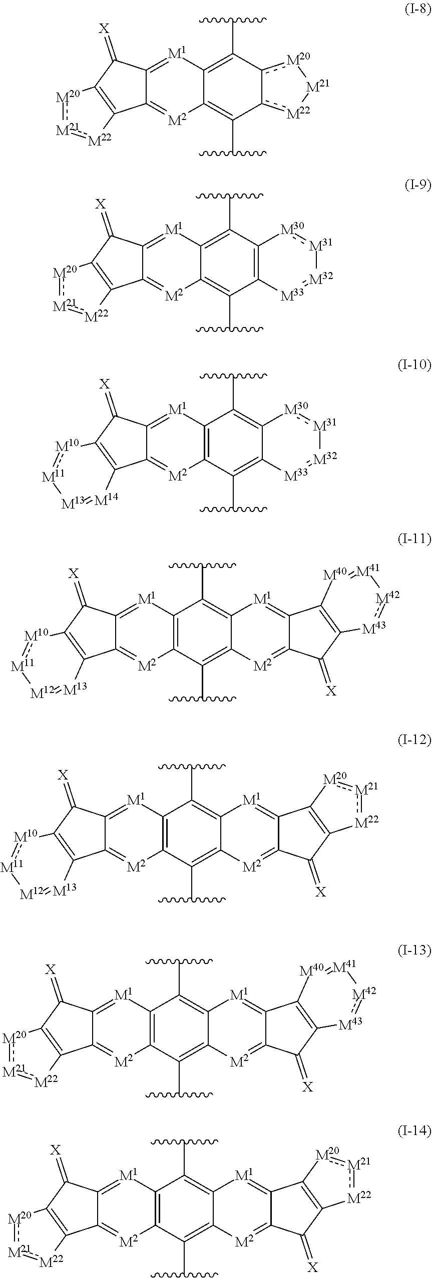

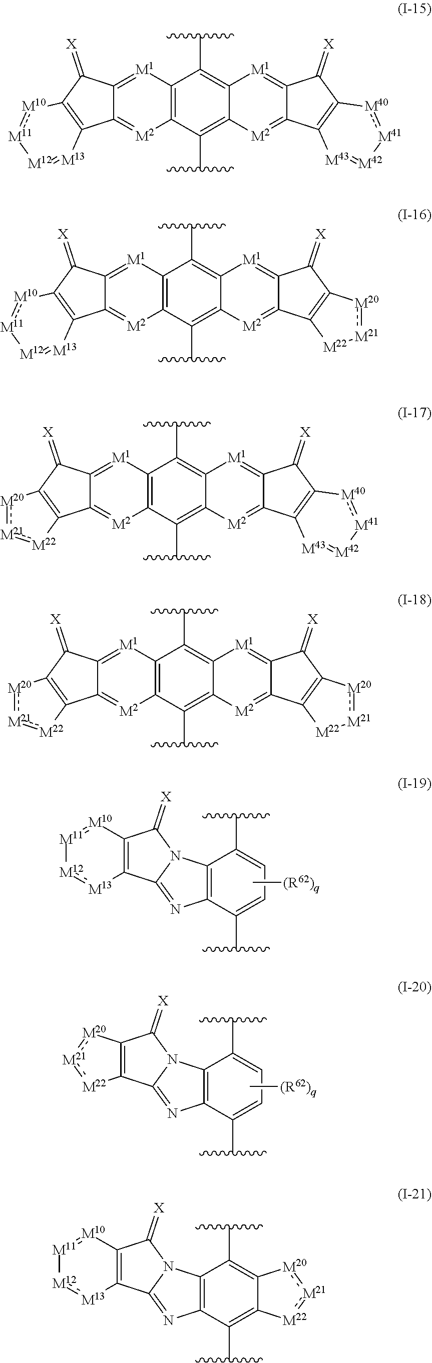

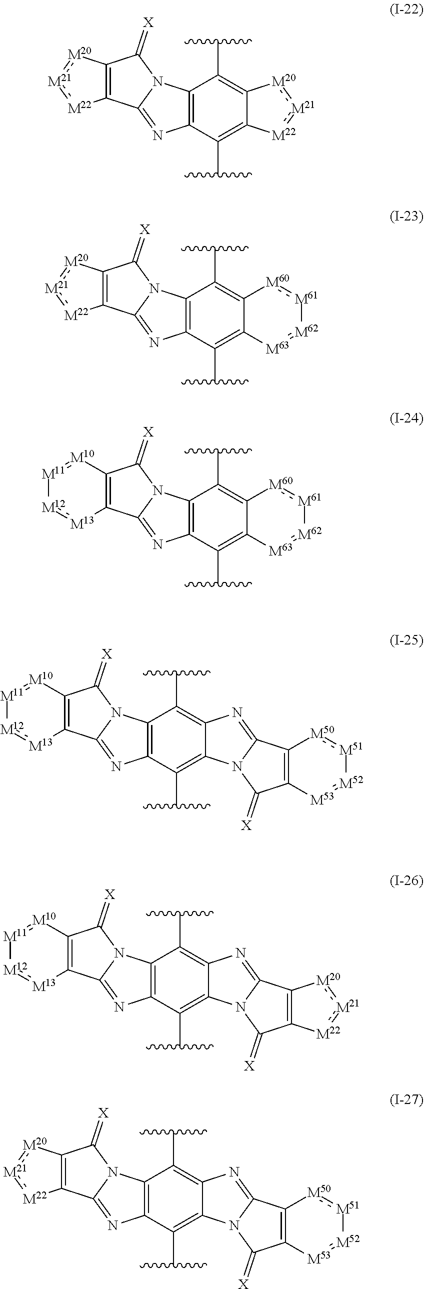

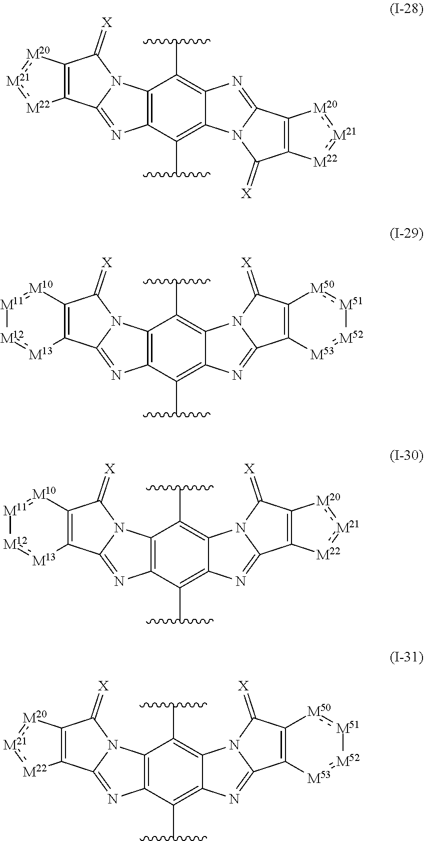

wherein
M¹, M², M³ and M⁴, is independently CR⁶¹ or N wherein R⁶¹ in each occurrence is a H or a substituent;
M¹⁰, M¹¹, M¹², M¹³, M²⁰, M²¹, M²², M³⁰, M³¹, M³², M³³, M⁴⁰, M⁴¹, M⁴², M⁴³, M⁵⁰, M⁵¹, M⁵², and M⁵³ is independently N, S, O or CR⁶¹, wherein R⁶¹ in each occurrence is a H or a substituent and with the proviso that a S or O is not adjacent to another S or O;
X is independently an electron withdrawing group; and
q is 0, 1, 2, 3 or 4.

Preferably, each unit of formula (I) is bound directly to at least one electron-donating unit D.

Preferably, Ar is a substituted or unsubstituted benzene or 5- or 6-membered heteroaromatic ring consisting of N and C ring atoms.

Preferably no more than 2 ring atoms of Ar are N atoms.

Preferably, Ar is selected from benzene, pyridine, and pyridazine.

More preferably, Ar is selected from benzene and pyridine.

Preferably, Ar¹ is a substituted or unsubstituted 5- or 6-membered heteroaromatic ring consisting of N and C ring atoms; consisting of N, C and O ring atoms; or consisting of N, C and S ring atoms.

Preferably no more than 2 ring atoms of Ar¹ are N atoms.

Optionally, no more than 1 ring atom of Ar¹ is an O or S atom.

Preferably, Ar¹ is selected from imidazole, pyridine, thiazine, pyrazine, and oxazine.

More preferably, Ar¹ is selected from imidazole and pyrazine.

Preferably, Ar² where present is as described for Ar¹ with the proviso that when Ar² is a 5-membered ring, Ar² is selected from imidazole and thiadiazole.

Preferably Ar³ is a 5-membered carbocyclic ring.

Preferably Ar⁴, where present is a 5-membered carbocyclic ring.

Preferably, Ar⁵ is independently a substituted or unsubstituted monocyclic or polycyclic group containing at least one aromatic or heteroaromatic ring, wherein the heteroaromatic ring consists of N and C ring atoms; consists of N, C and S ring atoms; or consists of N, C and O ring atoms.

Preferably no more than 2 ring atoms of Ar⁵ are N atoms.

Optionally, no more than 1 ring atom Ar⁵ is an O or S atom.

Preferably, Ar⁵ is selected from benzene, pyrrole, pyrazole, imidazole, oxazole, thiazole, pyridine, thiazine, a diazine including pyrimidine, pyridazine, pyrazine, thiadiazole, oxadiazole, oxazine, and triazole.

Preferably, Ar⁵ is selected from benzene, thiadiazole, triazole and a diazine for example pyrimidine, pyridazine or pyrazine.

More preferably, Ar⁵ is benzene.

Preferably, Ar⁶ where present is selected from groups as defined for Ar⁵.

Optionally, each R⁶⁰ and R⁶² is independently selected from H or a substituent described with respect to R⁶¹. Preferably each R⁶⁰ and R⁶² is independently selected from H, F, Cl, CN; C₁₋₂₀ alkyl wherein one or more H atoms may be replaced by F; unsubstituted phenyl; or phenyl substituted with one or more substituents selected from F and C₁₋₁₂ alkyl groups wherein one or more H atoms may be replaced with F.

Preferably, each electron-withdrawing group X is independently selected from O, S and NX⁷⁰ wherein X⁷⁰ is CN or COOR⁸⁰; and CX¹⁰X¹¹ wherein X¹⁰ and X¹¹ are each independently F, Cl, Br, CN, NO₂, CF₃, or COOR⁸⁰, wherein R⁸⁰ is H or a substituent, preferably a C₁₋₂₀ hydrocarbyl group, and preferably each of X¹⁰ and X¹¹ is F.

Optionally, each electron-withdrawing group is NX⁷⁰, wherein X⁷⁰ is selected from C₁₋₂₀alkyl wherein one or more non-adjacent, non-terminal C atoms may be replaced with O, S, COO or CO and one or more H atoms of the alkyl may be replaced with F; phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F; and a heteroaromatic group which is unsubstituted or substituted with one or more substituents.

Preferably, each electron-withdrawing group X is independently selected from O, S and NX⁷⁰ wherein X⁷⁰ is CN, COOR⁸⁰; C₁ to C₂₀ alkyl chain where any non-terminal C can be replaced by O or S, substituted or unsubstituted 5- or 6-membered aromatic or heteroaromatic ring; and CX¹⁰X¹¹ wherein X¹⁰ and X¹¹ are each independently selected from F, Cl, Br, CN, NO₂, CF₃, and COOR⁸⁰, wherein R⁸⁰ is H or a substituent, preferably a C₁₋₂₀ hydrocarbyl group, and preferably each of X¹⁰ and X¹¹ is F.

Preferably, each electron-withdrawing group X is independently selected from O and CX¹⁰X¹¹ wherein X¹⁰ and X¹¹ are each independently CN or COOR⁸⁰.

More preferably, each electron-withdrawing group X is independently selected from O and CX¹⁰X¹¹ wherein X¹⁰ and X¹¹ are each CN.

In a preferred embodiment, Ar is an optionally substituted benzene or a 6-membered heteroaromatic ring containing N and C atoms; Ar¹ is a 5- or 6-membered heteroaromatic ring containing N and C atoms; Ar² is an optionally substituted 5- or 6-membered heteroaromatic ring or is absent; Ar³ is a 5- or 6-membered ring; Ar⁴ is a 5- or 6-membered ring or is absent; Ar⁵ is an optionally substituted monocyclic or polycyclic group containing at least one aromatic or heteroaromatic ring; Ar⁶ is an optionally substituted monocyclic or polycyclic group containing at least one aromatic or heteroaromatic ring or is absent; and each X is independently an electron withdrawing group, bound to the C atoms of Ar³ and Ar⁴; and wherein the material further comprises a conjugated electron-donating unit D of formula (II).

In a more preferred embodiment, Ar is benzene; Ar¹ is a 6-membered heteroaromatic ring containing N and C atoms; Ar² is a substituted 6-membered heteroaromatic ring; Ar³ is a 5-membered ring; Ar⁵ is monocyclic containing one aromatic ring; and X is an electron withdrawing group bound to the C atom of Ar³; and wherein the material further comprises a conjugated electron-donating unit D of formula (II).

In a preferred embodiment, Ar is benzene; Ar¹ is a 6-membered heteroaromatic ring containing N and C atoms; Ar² is an optionally substituted 5- or 6-membered heteroaromatic ring or is absent; Ar³ is a 5-membered ring; Ar⁴ is a 5-membered ring or is absent; Ar⁵ is an optionally substituted monocyclic or polycyclic group containing at least one aromatic or heteroaromatic ring; Ar⁶ is independently an optionally substituted monocyclic or polycyclic group containing at least one aromatic or heteroaromatic ring or is absent; and each X is independently an electron withdrawing group bound to the C atoms of Ar³ and/or Ar⁴; and wherein the material further comprises a conjugated electron-donating unit D of formula (II).

In a preferred embodiment, Ar is benzene; Ar¹ is a 6-membered heteroaromatic ring containing N and C atoms; Ar² 5-membered heteroaromatic ring; Ar³ is a 5-membered ring; Ar⁵ is monocyclic group containing one aromatic ring; and X an electron withdrawing group bound to the C atom of Ar³; and wherein the material further comprises a conjugated electron-donating unit D of formula (II).

In a preferred embodiment, Ar is benzene; Ar¹ is a 5-membered heteroaromatic ring containing N and C atoms; Ar² is an optionally substituted 5- or 6-membered heteroaromatic ring or is absent; Ar³ is a 5-membered ring; Ar⁴ is a 5-membered ring or is absent; Ar⁵ is an optionally substituted monocyclic or polycyclic group containing at least one aromatic or heteroaromatic ring; Ar⁶ is an optionally substituted monocyclic or polycyclic group containing at least one aromatic or heteroaromatic ring or is absent; and each X is independently an electron withdrawing group bound to the C atoms of Ar³ and/or Ar³; and wherein the material further comprises a conjugated electron-donating unit D of formula (II).

Exemplary units of formula (I) include the following which may be unsubstituted or substituted with one or more substituents R⁶¹ as described above:

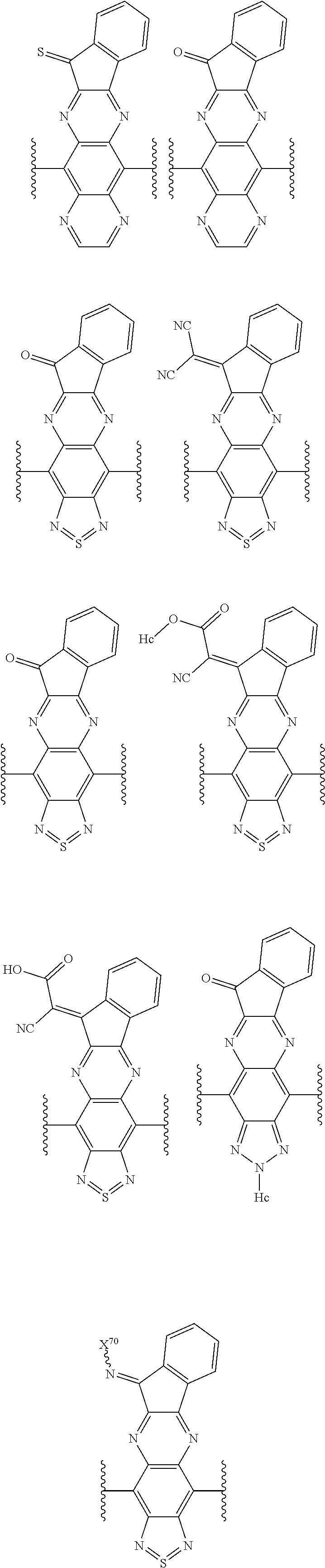

wherein Hc is a C₁₋₂₀ hydrocarbyl group, e.g. a C₁₋₂₀ alkyl group, an unsubstituted phenyl or phenyl substituted with one or more C₁₋₁₂ alkyl groups.

In some embodiments, the material comprising the unit of formula (I) has an absorption peak greater than 750 nm.

In some embodiments, the material comprising the unit of formula (I) has an absorption peak greater than 900 nm.

In some embodiments, the material comprising the unit of formula (I) has an absorption peak greater than 1100 nm.

In some embodiments the material comprising the unit of formula (I) has an absorption peak in the range of 750-2000 nm, between 750-1400 nm, between 750-900 nm or 900-2000 nm.

Unless stated otherwise, absorption spectra of materials as described herein are measured using a Cary 5000 UV-VIS-NIR Spectrometer. Measurements were taken from 175 nm to 3300 nm using a PbSmart NIR detector for extended photometric range with variable slit widths (down to 0.01 nm) for optimum control over data resolution.

Absorption data are obtained by measuring the intensity of transmitted radiation through a solution sample. Absorption intensity is plotted vs. incident wavelength to generate an absorption spectrum. A method for measuring absorption, may comprise measurement of a 15 mg/ml solution in a quartz cuvette compared to a spectrum of the solvent only in a cuvette.

Unless stated otherwise, absorption data as provided herein is as measured in toluene solution.

The electron donor (p-type) material has a HOMO deeper (further from vacuum) than a LUMO of the electron acceptor (n-type) material. Optionally, the gap between the HOMO level of the p-type donor material and the LUMO level of the n-type acceptor material is less than 1.4 eV. Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCI reference electrode.

Ferrocene is added directly to the existing cell at the end of the experiment for calculation purposes where the potentials are determined for the oxidation and reduction of ferrocene versus Ag/AgCI using cyclic voltammetry (CV).

The sample is dissolved in toluene (3 mg/ml) and spun at 3000 rpm directly on to the glassy carbon working electrode.

$$LUMO = 4.8 - E ferrocene ( peak to peak average ) - E reduction of sample ( peak maximum ) . \\ HOMO = 4.8 - E ferrocene ( peak to peak average ) + E oxidation of sample ( peak maximum ) .$$

A typical SWV experiment runs at 15 Hz frequency; 25 mV amplitude and 0.004 V increment steps. Results are calculated from 3 freshly spun film samples for both the HOMO and LUMO data.

In some embodiments, the bulk heterojunction layer contains only one electron donor material and only one electron acceptor material, at least one of the donor and acceptor comprising an electron-accepting unit of formula (I).

In some embodiments, the bulk heterojunction layer contains two or more electron donor materials and/or two or more electron acceptor materials.

In some embodiments, the weight of the donor material(s) to the acceptor material(s) is from about 1:0.5 to about 1:2, preferably about 1: 1.1 to about 1:2.

In some embodiments, the material comprising the group of formula (I) is a non-polymeric compound containing at least one unit of formula (I), optionally 1, 2 or 3 units of formula (I) and at least on electron-donating unit D. Preferably, the non-polymeric compound has a molecular weight of less than 5,000 Daltons, optionally less than 3,000 Daltons. Preferably, the non-polymeric compound contains no more than 3 groups of formula (I).

In some embodiments, the material comprising the group of formula (I) is a polymer comprising a repeat unit of formula (I) and an electron-donating repeat unit, more preferably alternating electron-accepting repeat units of formula (I) and electron-donating repeat units.

Preferably, the polystyrene-equivalent number-average molecular weight (Mn) measured by gel permeation chromatography of the polymer is in the range of about 5×10³ to 1×10⁸, and preferably 1×10⁴ to 5×10⁶. The polystyrene-equivalent weight-average molecular weight (Mw) of the polymer may be 1×10³ to 1×10⁸, and preferably 1×10⁴ to 1×10⁷.

In some embodiments the polymer may be part of a composition comprising or consisting of an electron-accepting (n-type) material and an electron-donating (p-type) material wherein the polymer is the electron-donating material. The composition may comprise one or more further materials, e.g. one or more further electron-donating materials and/or one or more further electron-accepting materials.

Preferably, each unit of formula (I) is bound directly to at least one electron-donating unit D.

A non-polymeric compound comprising a unit of formula (I) may have formula (Ia)-(Id):

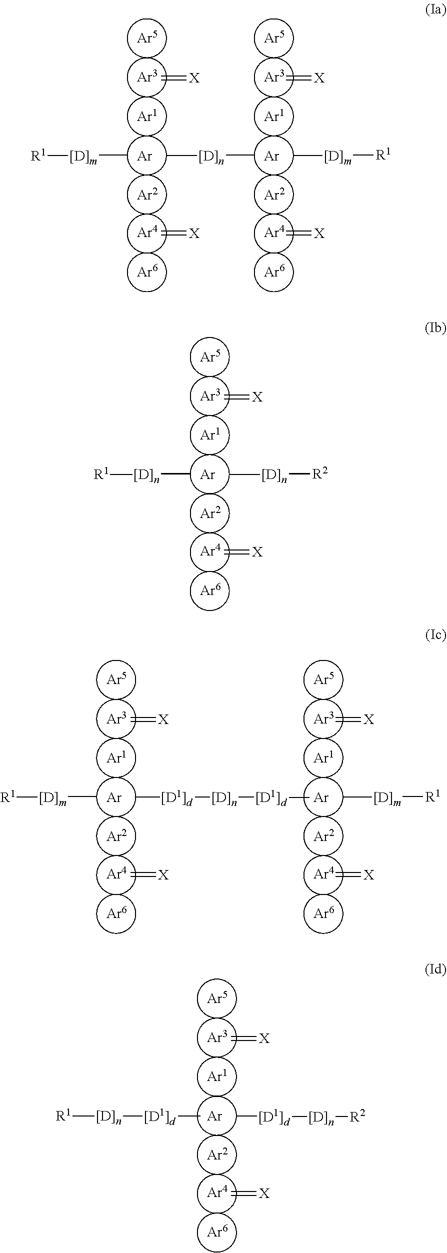

wherein n is at least 1, optionally 1, 2 or 3; m is 0, 1, 2 or 3; d is 0, 1 or 2; D in each occurrence is independently a conjugated electron-donating unit D of formula (II); R¹ and R² independently in each occurrence is H or a substituent; Ar and Ar¹-Ar⁶ and X are as described above; and D¹ is a conjugated bridge unit which is different from D.

Preferably, R¹ in each occurrence is the same; R² in each occurrence is the same.

Optionally, R¹ and R² are each independently selected from the group consisting of H; an electron withdrawing group including but not limited to F, CN, and NO₂; C₁₋₂₀ alkyl wherein one or more non-adjacent, non-terminal C atoms may be replaced with O, S, COO or CO and one or more H atoms of the alkyl may be replaced with F; phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F.

In some embodiments the polymer comprising repeat units of formula (I) may contain the repeating structure of formula (V), comprising the repeat unit of formula (I) and an adjacent conjugated electron-donating repeat unit D of formula (II):

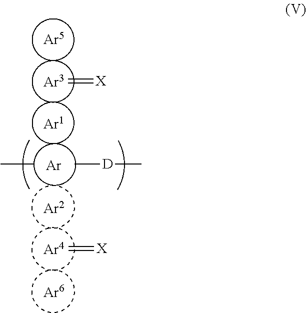

In some embodiments the polymer comprising repeat units of formula (I) may contain the repeating structure of formula (Va), comprising the repeat unit of formula (I) and an adjacent conjugated electron-donating repeat unit D of formula (II) and may further comprise an electron-donating repeat structure D¹ which is different from D:

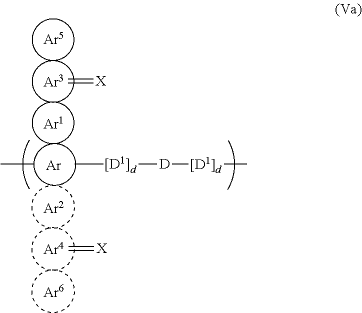

wherein Ar and Ar¹-Ar⁶, X, D, D¹ and d are as described above.

For an electron donor material or electron acceptor material containing an electron accepting unit of formula (I) and a conjugated electron-donating unit D of formula (II) the, or each, unit of formula (I) has a LUMO level that is deeper (i.e. further from vacuum) than the, or each, electron-donating unit, preferably at least 1 eV deeper. The LUMO levels of an electron-donating unit and an electron-accepting unit of formula (I) may be as determined by modelling, the LUMO level of D-H or H-D-H and H-[Formula (I)]-H, respectively, i.e. by replacing the bond or bonds between D and Formula (I) with a bond or bonds to a hydrogen atom.

Preferably, a model compound of formula H-[Formula (I)]-H containing one or more electron-withdrawing groups deepens the LUMO by at least 0.2 eV as compared to the case where the electron-withdrawing groups are absent.

### Additional Electron-Donating unit

In some embodiments, the conjugated electron-donating unit D of formula (II) is the only electron-donating unit present. In some embodiments the material contains an electron donating unit other than formula (II).

Optionally, the additional electron-donating unit is a bridging unit disposed between the groups of formula (I) and (II) and is preferably in direct contact with the groups of formula (I) and (II).

Optionally, such additional electron-donating units are in each occurrence a monocyclic or polycyclic heteroaromatic group which contains at least one furan or thiophene and which may be unsubstituted or substituted with one or more substituents. Optionally, the additional electron donating units may be selected from formulae (IIa)-(IIp):

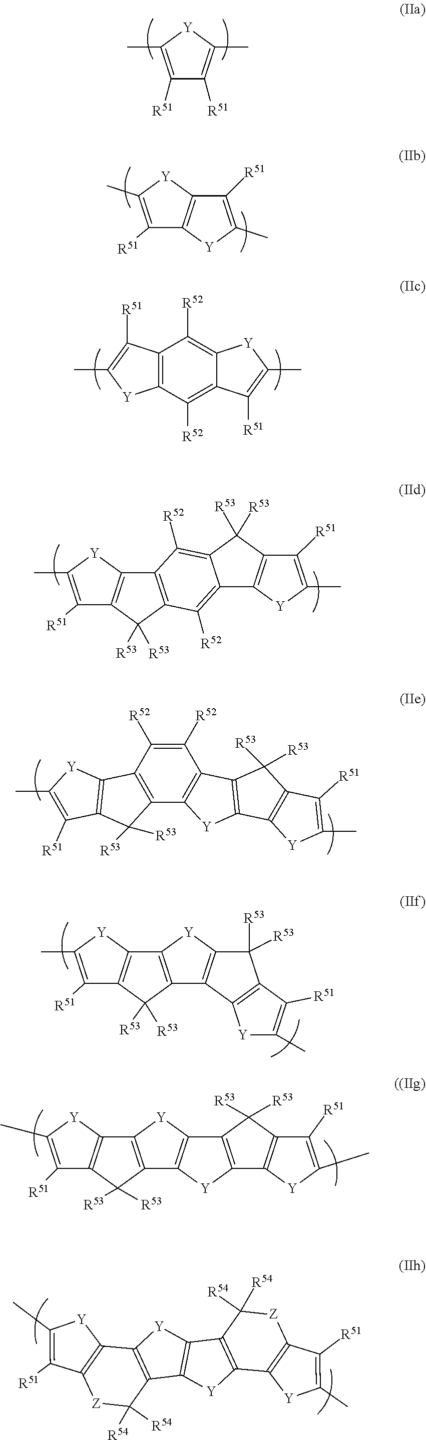

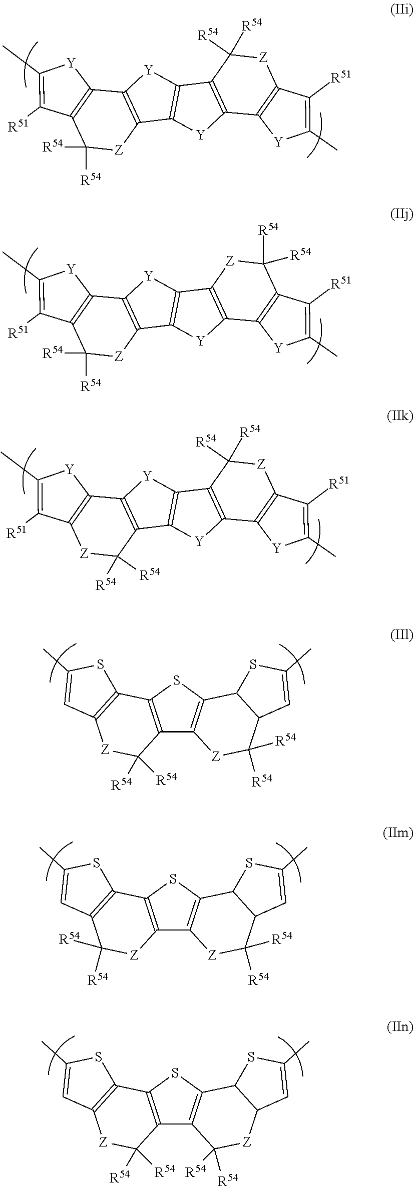

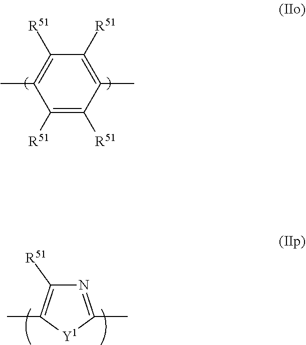

wherein Y and Y¹ in each occurrence is independently O, S, or NR⁵⁵, preferably S; Z in each occurrence is O, NR⁵⁵, or C(R⁵⁴)₂; R⁵⁰, R⁵¹, R⁵² and R⁵⁴ independently in each occurrence is H or a substituent wherein R⁵⁰ groups may be linked to form a ring; and R⁵³ and R⁵⁵ independently in each occurrence is a substituent.

The group of formula (IIa) is preferred. The group of formula (IIa) in which Y is S is particularly preferred.

Optionally, R⁵⁰, R⁵¹ and R⁵² independently in each occurrence are selected from H; F; C₁₋₂₀ alkyl wherein one or more non-adjacent, non-terminal C atoms may be replaced with O, S, COO or CO and one or more H atoms of the alkyl may be replaced with F; and an aromatic or heteroaromatic group Ar³ which is unsubstituted or substituted with one or more substituents.

In some embodiments, Ar³ may be an aromatic group, e.g. phenyl.

The one or more substituents of Ar³, if present, may be selected from C₁₋₁₂ alkyl wherein one or more non-adjacent, non-terminal C atoms may be replaced with O, S, COO or CO and one or more H atoms of the alkyl may be replaced with F.

Preferably, each R⁵⁴ is selected from the group consisting of:
H;
linear, branched or cyclic C₁₋₂₀ alkyl wherein one or more non-adjacent, non-terminal C atoms may be replaced by O, S, NR⁷, CO or COO wherein R⁷ is a C₁₋₁₂ hydrocarbyl and one or more H atoms of the C₁₋₂₀ alkyl may be replaced with F; and
a group of formula (Ak)u-(Ar⁴)v wherein Ak is a C₁₋₁₂ alkylene chain in which one or more C atoms may be replaced with O, S, CO or COO; u is 0 or 1; Ar⁴ in each occurrence is independently an aromatic or heteroaromatic group which is unsubstituted or substituted with one or more substituents; and v is at least 1, optionally 1, 2 or 3.

Preferably, each R⁵¹ is H.

Optionally, R⁵³ independently in each occurrence is selected from C₁₋₂₀ alkyl wherein one or more non-adjacent, non-terminal C atoms may be replaced with O, S, COO or CO and one or more H atoms of the alkyl may be replaced with F; and phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F.

Preferably, R⁵⁵ is a C₁₋₃₀ hydrocarbyl group.

### Polymer Synthesis and Monomers

A polymer as described herein may be formed by polymerising a monomer for forming electron-donating repeat unit D and a monomer for forming the electron-accepting repeat unit of formula (I). If a conjugated bridge unit is present, then one of these monomers further contains the unit D¹. The polymerisation method includes, without limitation, methods for forming a carbon-carbon bond between an aromatic carbon atom of an electron-donating unit D or D¹ and an aromatic carbon atom of an electron-accepting unit (I).

In some embodiments, formation of the polymer comprises polymerisation of a monomer of formula (Xa) and a monomer of formula (Xb):

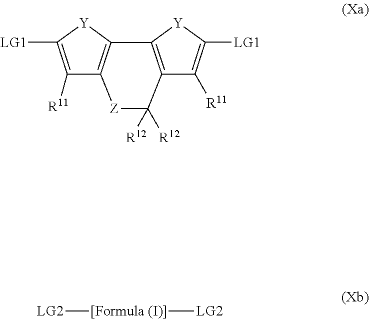

In some embodiments, formation of the polymer comprises polymerisation of a monomer of formula (Xc) and a monomer of formula (Xb):

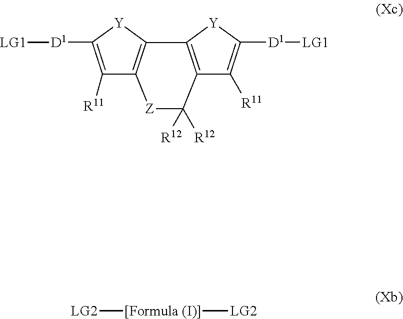

In some embodiments, formation of the polymer comprises polymerisation of a monomer of formula (Xa) and a monomer of formula (Xd):

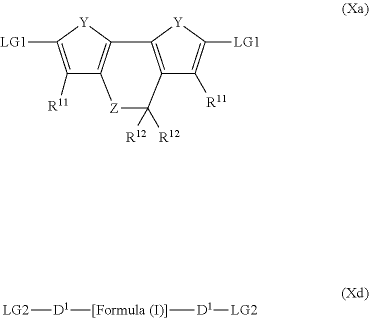

R¹¹, R¹², Y and Z are as described above.

LG1 is a first leaving group bound to an aromatic carbon atom.

LG2 is a second leaving group bound to an aromatic carbon atom which is different from LG1.

A carbon-carbon bond is formed during polymerisation between aromatic carbon atoms to which LG1 and LG2 are bound.

It will be understood that a repeat unit as described anywhere herein may be formed from a monomer comprising or consisting of the repeat unit and leaving groups. For example, polymerisation of Formula (Xd) forms a repeat unit including D1 and Formula (I) repeat units.

Optionally, LG1 is selected from one of group (a) and group (b), and LG2 is selected from the other of group (a) and group (b):
(a) halogen or —OSO₂R⁶ wherein R⁶ is an optionally substituted C₁₋₁₂ alkyl group or optionally substituted aryl group;
(b) boronic acid and esters thereof; and —SnR⁹₃ wherein R⁹ independently in each occurrence is a C₁₋₁₂ hydrocarbyl group.

Suitable polymerisation methods include, without limitation, Suzuki polymerisation and Stille polymerisation. Suzuki polymerisation is described in, for example, WO 00/53656.

In some embodiments, each LG1 may be one of: (i) a halogen or —OSO₂R⁶; or (ii), a boronic acid or ester, and each LG2 may be the other of (i) and (ii).

In some embodiments, each LG1 may be one of: (i) a halogen or —OSO₂R⁶; and (iii) —SnR⁹₃, and each LG2 may be the other of (i) and (iii).

Optionally, R⁶ in each occurrence is independently a C₁₋₁₂ alkyl group which is unsubstituted or substituted with one or more F atoms; or phenyl which is unsubstituted or substituted with one or more F atoms.

—OSO₂R⁶ is preferably tosylate or triflate.

Exemplary boronic esters have formula (VIII):

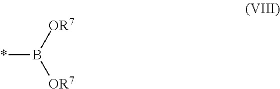

wherein R⁷ in each occurrence is independently a C₁₋₂₀ alkyl group, * represents the point of attachment of the boronic ester to an aromatic ring of the monomer, and the two groups R⁷ may be linked to form a ring which is unsubstituted or substituted with one or more substituents, e.g. one or more C₁₋₆ alkyl groups.

Optionally, R⁷ independently in each occurrence is selected from the group consisting of C₁₋₁₂ alkyl; unsubstituted phenyl; and phenyl substituted with one or more C₁₋₆ alkyl groups.

In a preferred embodiment, the two groups R⁷ are linked, e.g. to form:

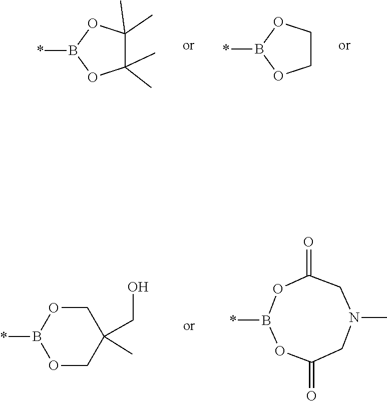

A halogen leaving group is preferably Br or I.

### Electron Donor Material

In the case where the material comprising the group of formula (I) is an electron-accepting material, it may be used with any electron donor material containing a group of formula (I) or any other electron donor material known to the person skilled in the art, including organic polymers and non-polymeric organic molecules.

In a preferred embodiment the electron donor material is an organic conjugated polymer, which can be a homopolymer or copolymer including alternating, random or block copolymers. Preferred are non-crystalline or semi-crystalline conjugated organic polymers. Further preferably the p-type organic semiconductor is a conjugated organic polymer with a narrow band gap, typically between 2.5 eV and 1.5 eV, preferably between 2.3 eV and 1.8 eV.

Optionally, the p-type donor has a HOMO level no more than 5.5 eV from vacuum level. Optionally, the p-type donor has a HOMO level at least 4.1 eV from vacuum level.

As exemplary p-type donor polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3-substituted thiophene), poly(3,4-bisubstituted thiophene), polyselenophene, poly(3-substituted selenophene), poly(3,4-bisubstituted selenophene), poly(bisthiophene), poly(terthiophene), poly(bisselenophene), poly(terselenophene), polythieno[2,3-b]thiophene, polythieno[3,2-b]thiophene, polybenzothiophene, polybenzo[1,2-b:4,5-b′]dithiophene, polyisothianaphthene, poly(monosubstituted pyrrole), poly(3,4-bisubstituted pyrrole), poly-1,3,4-oxadiazoles, polyisothianaphthene, derivatives and co-polymers thereof may be mentioned. Preferred examples of p-type donors are copolymers of polyfluorenes and polythiophenes, each of which may be substituted, and polymers comprising benzothiadiazole-based and thiophene-based repeating units, each of which may be substituted. It is understood that the p-type donor may also consist of a mixture of a plurality of electron donating materials.

Exemplary electron donor polymers comprising a repeat unit of formula (I) include polymers having a repeating structure selected from:

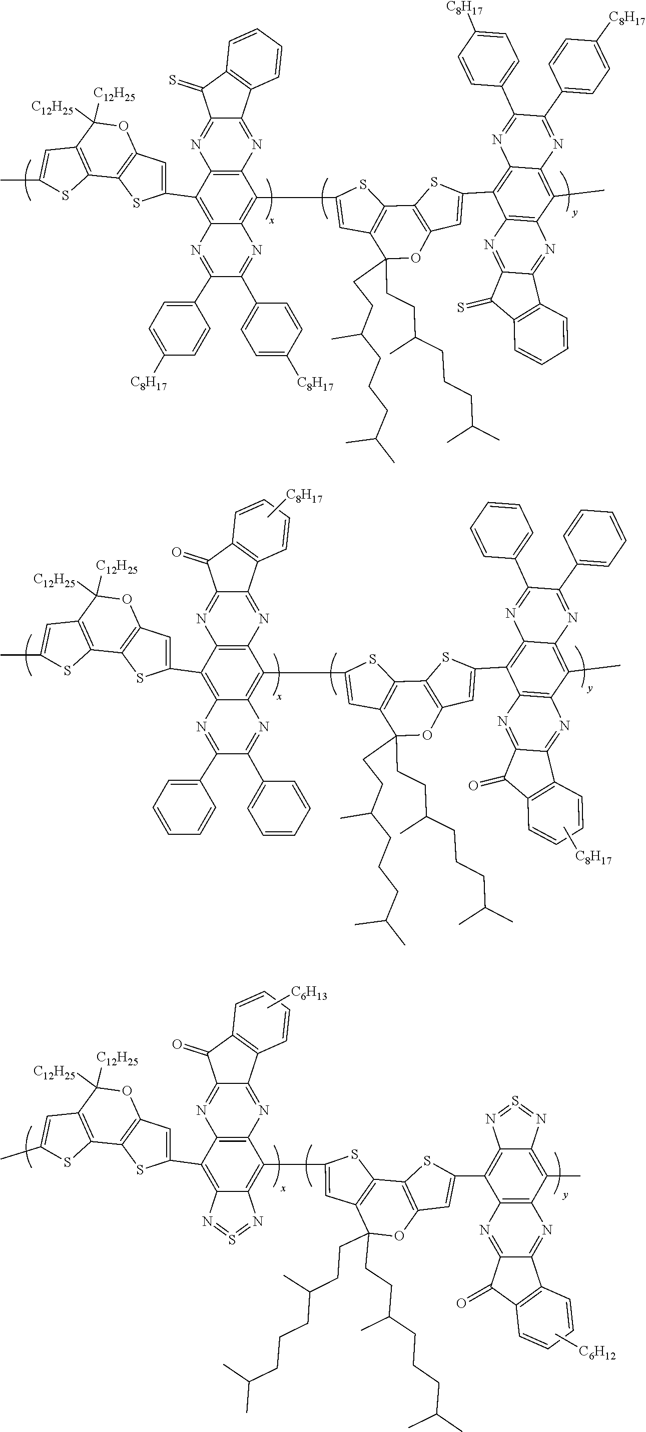

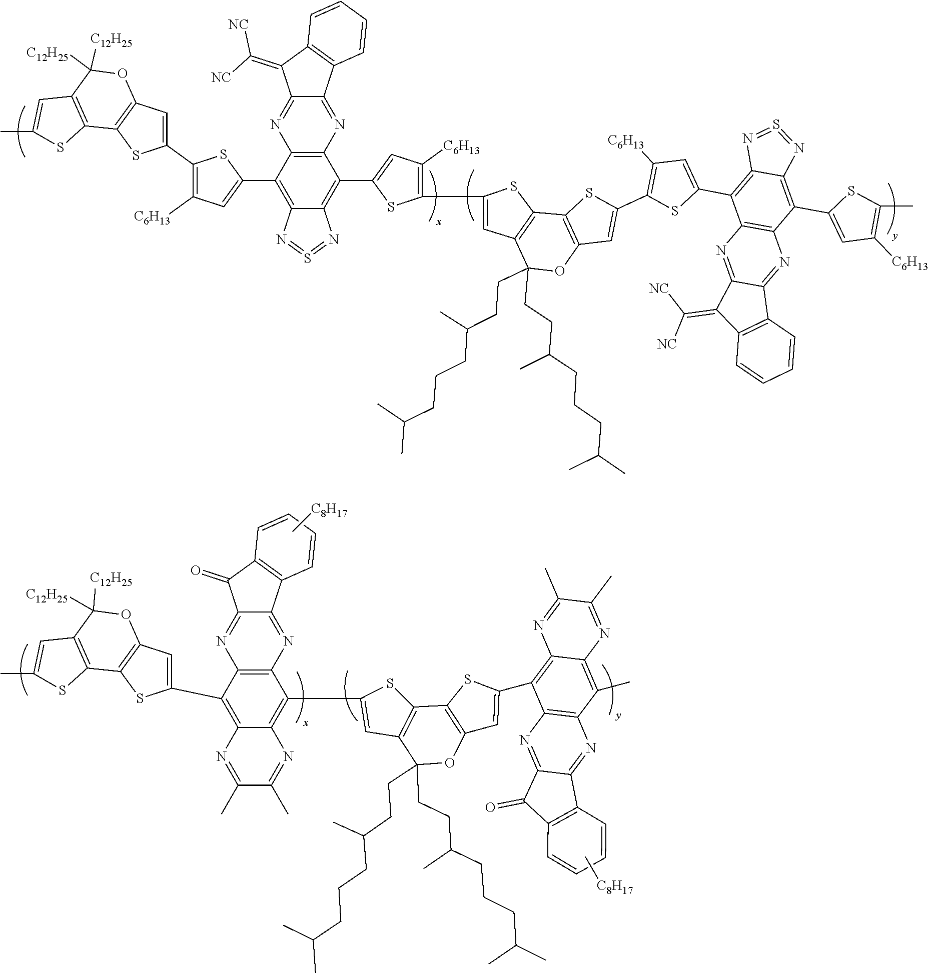

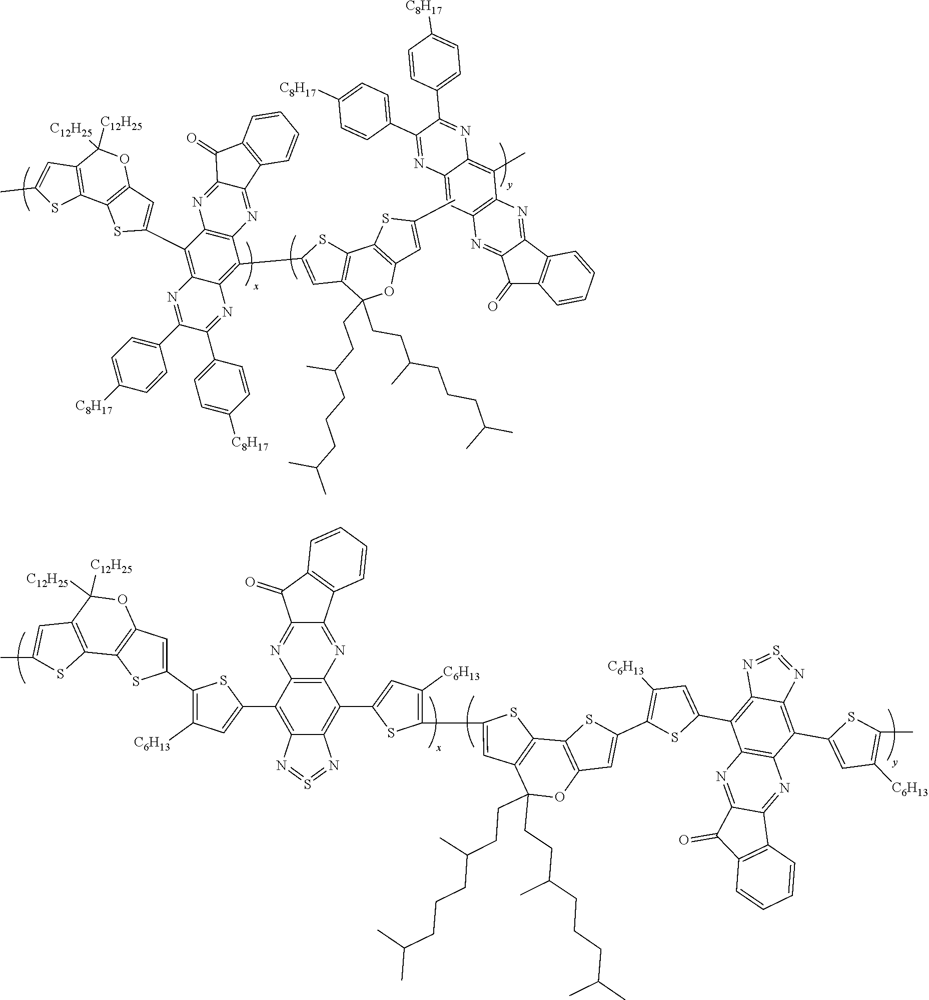

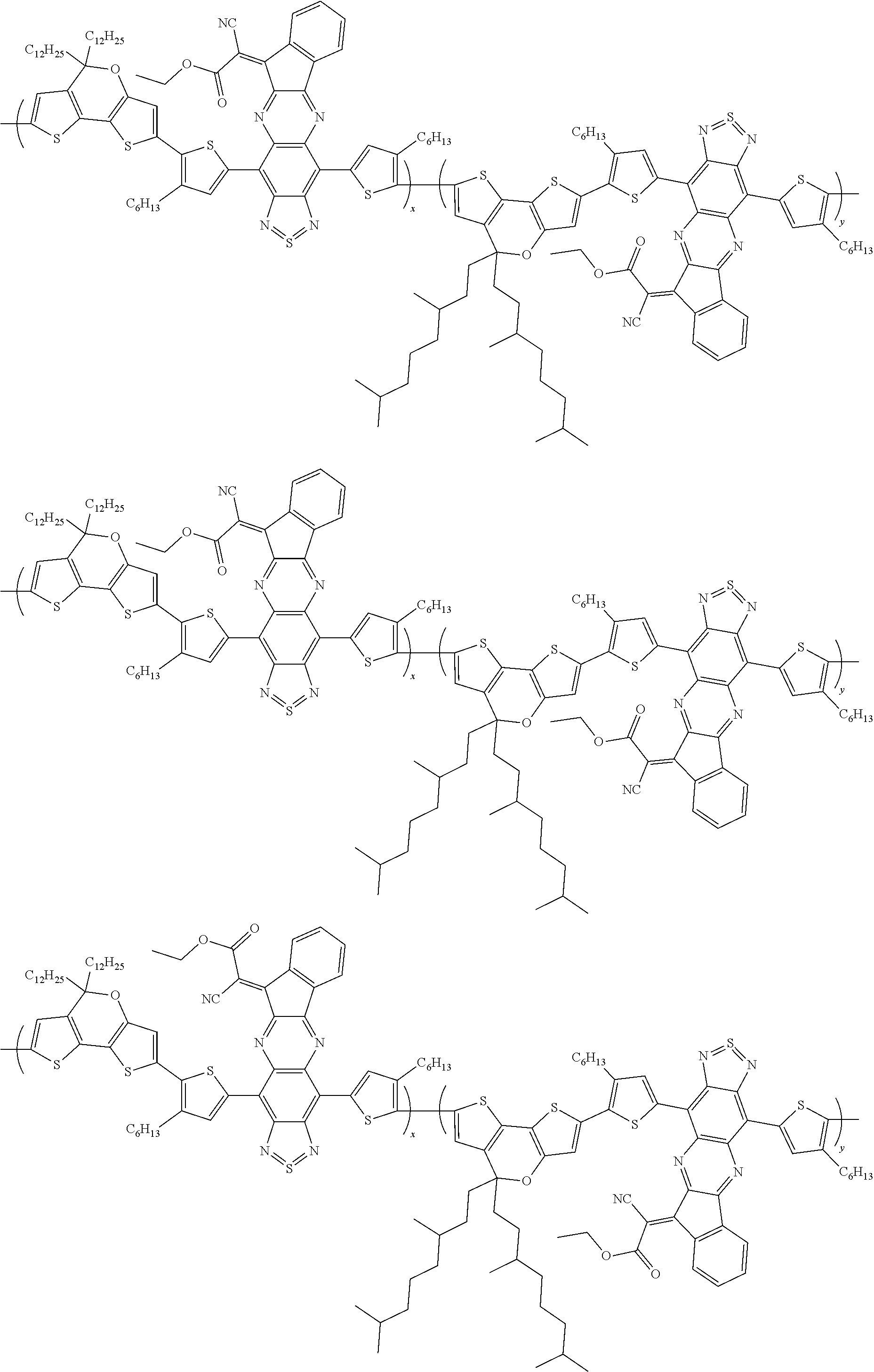

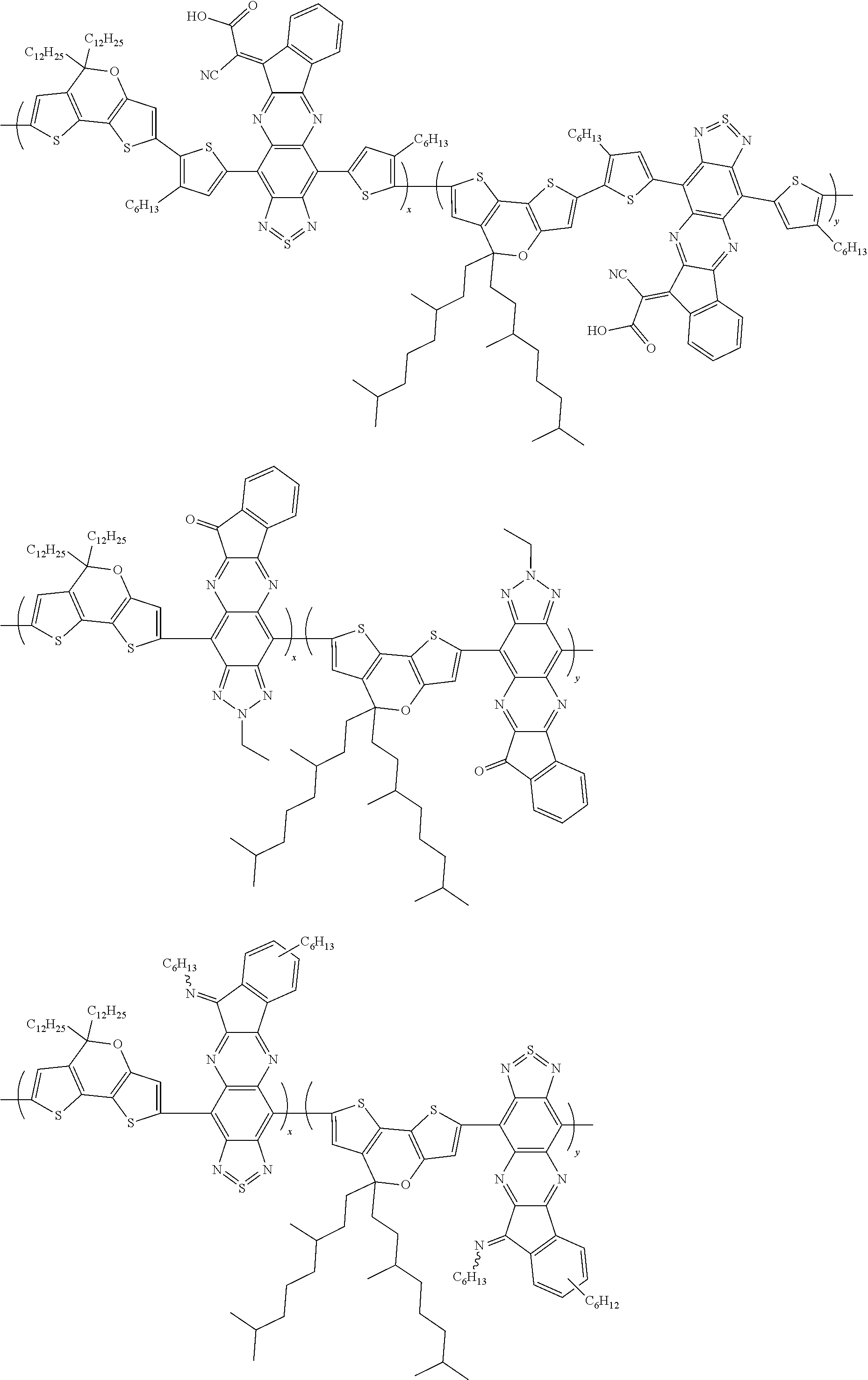

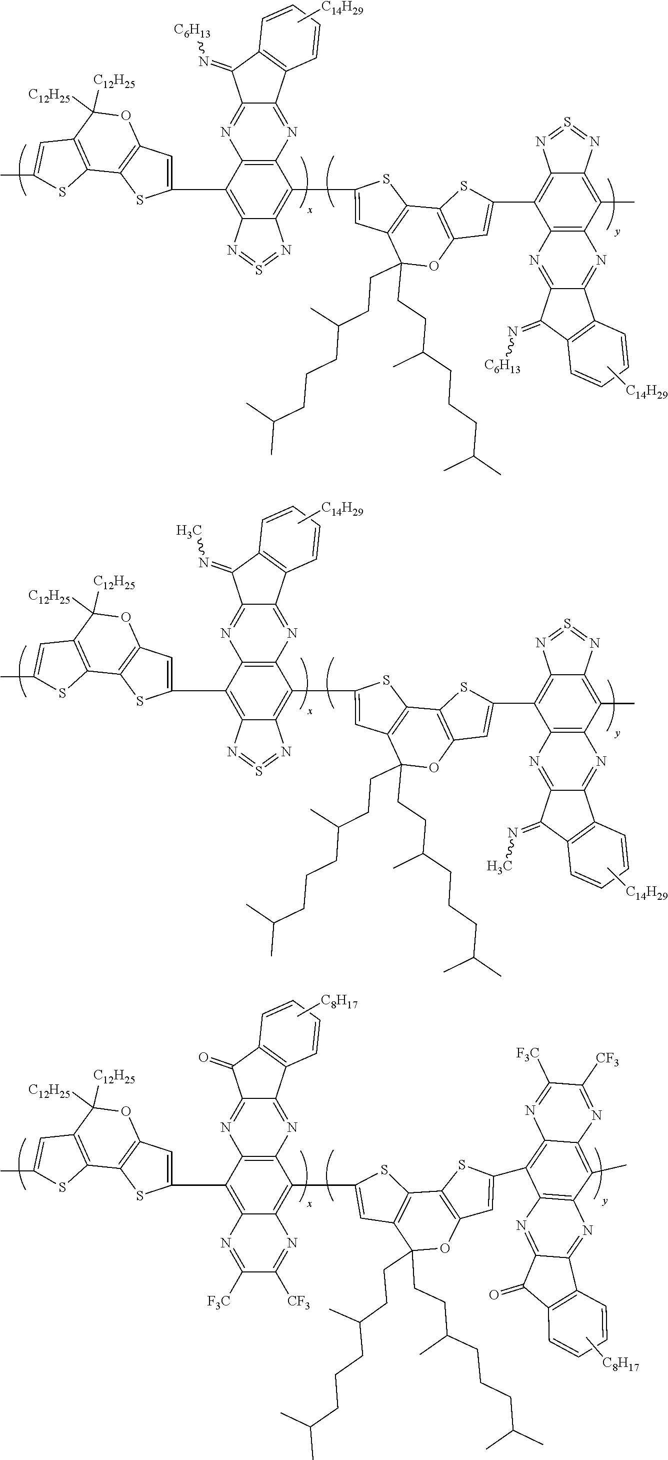

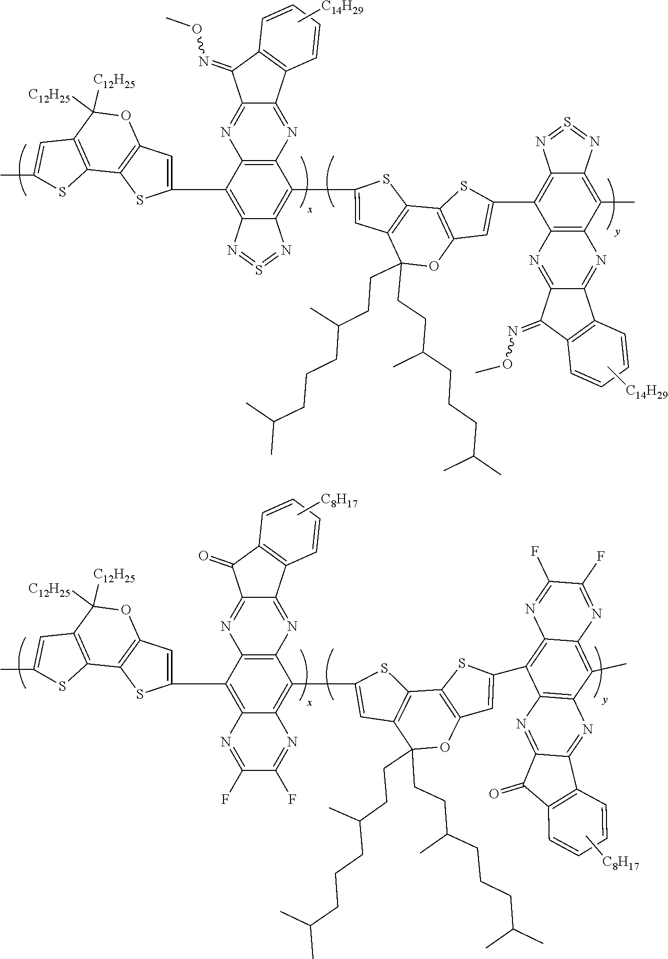

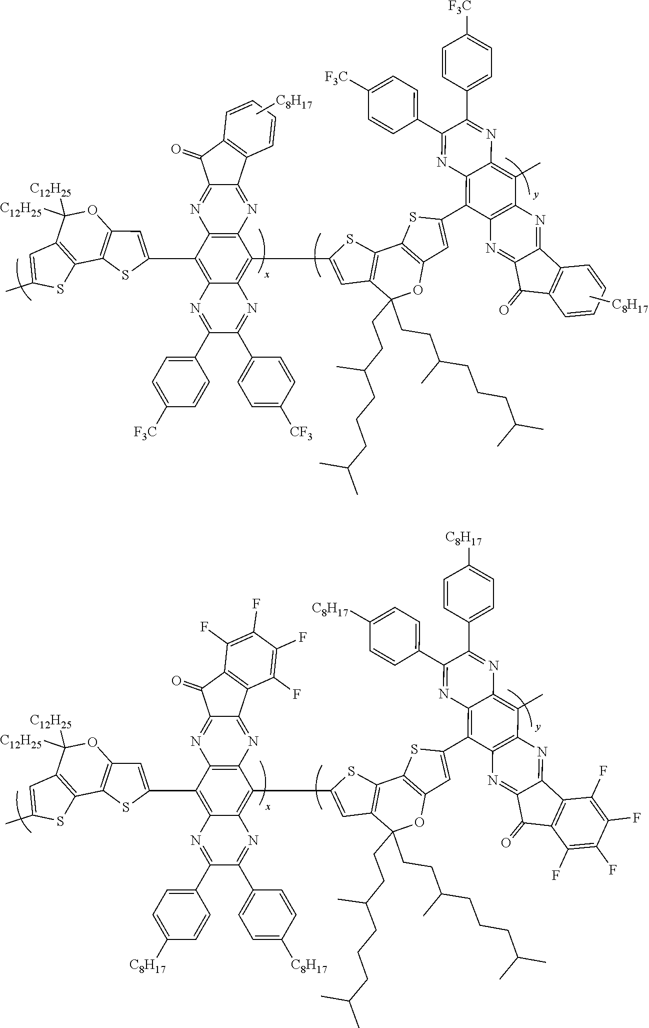

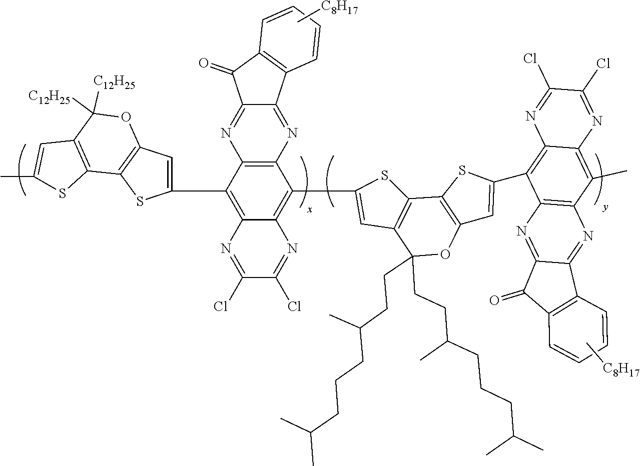

wherein x and y are each an amount of the illustrated repeating structures: x/(x+y)=1; 0≤x≤1; 0≤y≤1; and x+y=1.

Optionally, in the case where the electron donor polymer does not contain a repeat unit of formula (I), it comprises a repeat unit selected from repeat units of formulae:

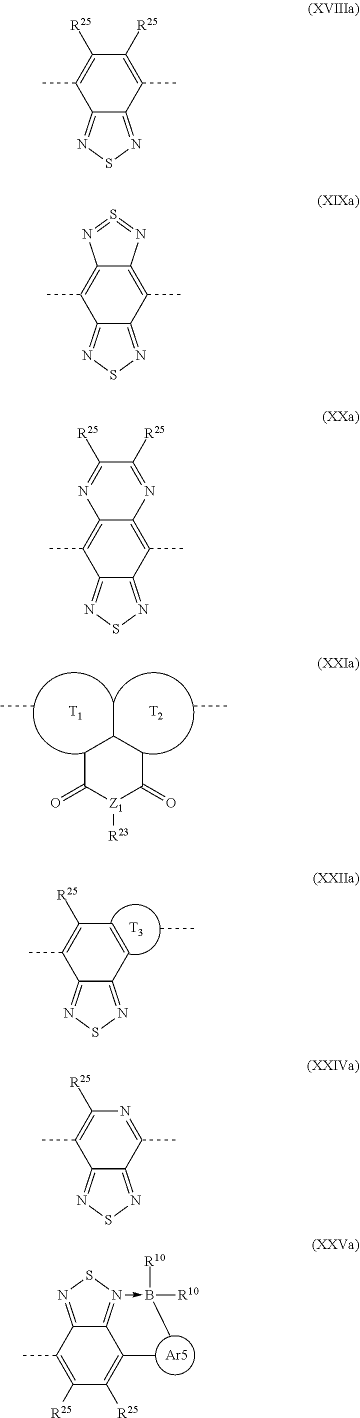

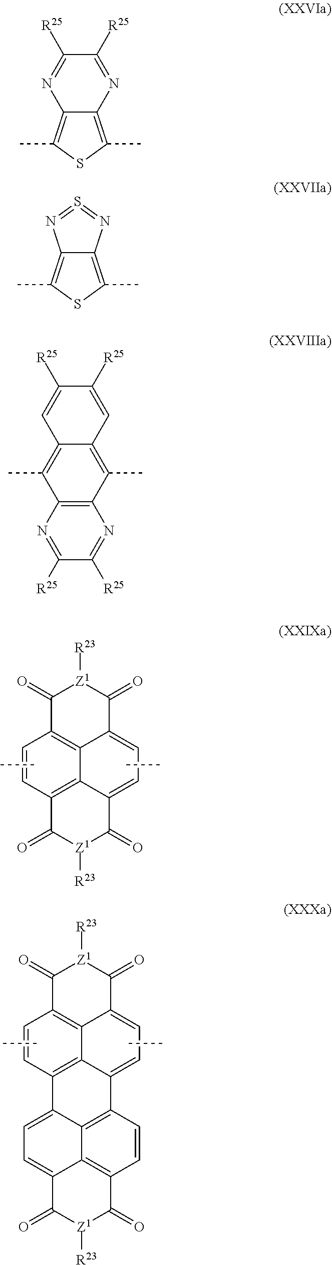

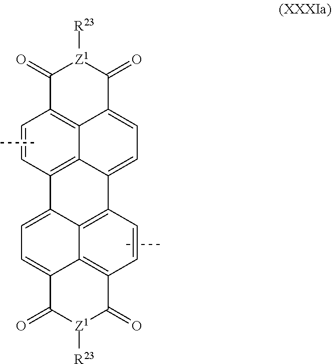

R²³ in each occurrence is a substituent, optionally C₁₋₁₂ alkyl wherein one or more non-adjacent, non-terminal C atoms may be replaced with O, S, COO or CO and one or more H atoms of the alkyl may be replaced with F.

R²⁵ in each occurrence is independently H; F; CN; NO₂; C₁₋₁₂ alkyl wherein one or more non-adjacent, non-terminal C atoms may be replaced with O, S, COO or CO and one or more H atoms of the alkyl may be replaced with F; an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO: or

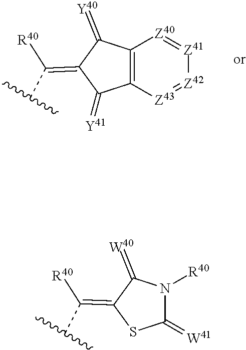

wherein Z⁴⁰, Z⁴¹, Z⁴² and Z⁴³ are each independently CR¹³ or N wherein R¹³ in each occurrence is H or a substituent, preferably a C₁₋₂₀ hydrocarbyl group;
Y⁴⁰ and Y⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰;
W⁴⁰ and W⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰; and
R⁴⁰ in each occurrence is H or a substituent, preferably H or a C₁₋₂₀ hydrocarbyl group.

Z¹ is N or P.

T¹, T² and T³ each independently represent an aryl or a heteroaryl ring, optionally benzene, which may be fused to one or more further rings. Substituents of T¹, T² and T³, where present, are optionally selected from non-H groups of R²⁵.

R¹⁰ in each occurrence is a substituent, preferably a C₁₋₂₀ hydrocarbyl group.

Ar⁵ is an arylene or heteroarylene group, optionally thiophene, fluorene or phenylene, which may be unsubstituted or substituted with one or more substituents, optionally one or more non-H groups selected from R²⁵.

Exemplary donor materials are disclosed in, for example, WO2013051676, the contents of which are incorporated herein by reference.

### Electron Acceptor Material

In the case where the material comprising the group of formula (I) is an electron donor material, it may be used with any electron-accepting material containing a group of formula (I) or any other electron-accepting material known to the person skilled in the art.

Exemplary electron-accepting materials are non-fullerene acceptors, which may or may not contain a unit of formula (I), and fullerenes. A composition containing the material comprising the group of formula (I) may comprise only one electron-accepting material or it may comprise two or more electron-accepting materials, for example at least one non-fullerene acceptor and at least one fullerene acceptor.

Exemplary electron-accepting compounds containing at least one unit of formula (I) include:

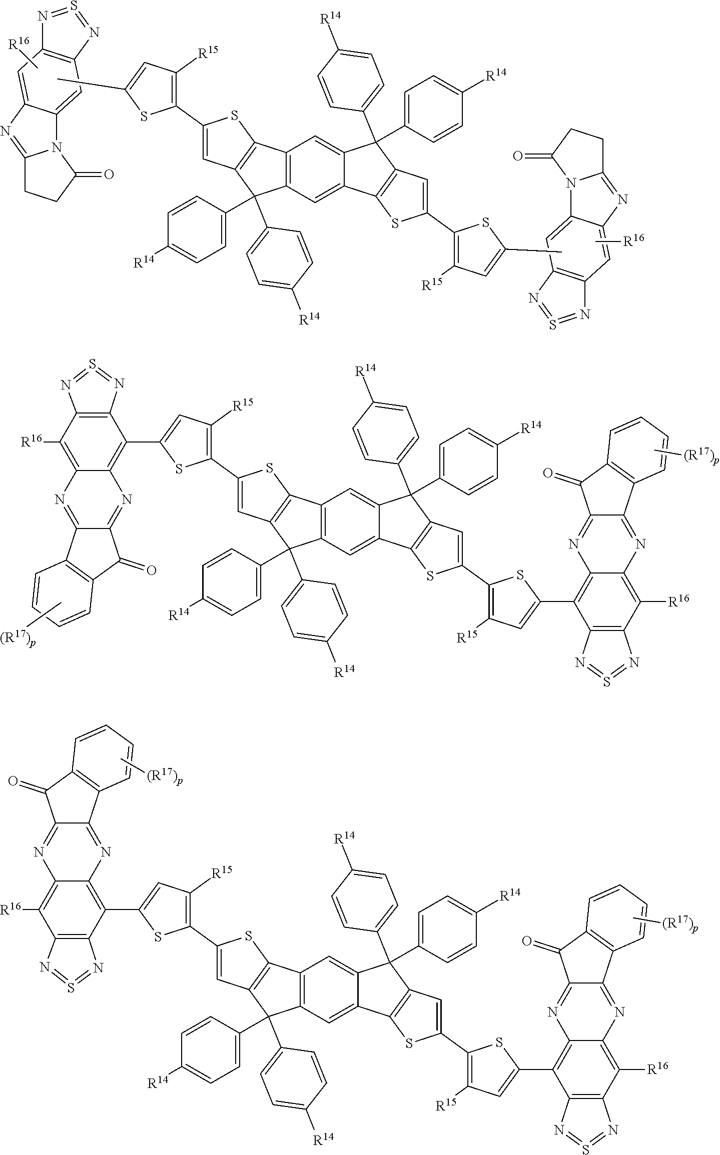

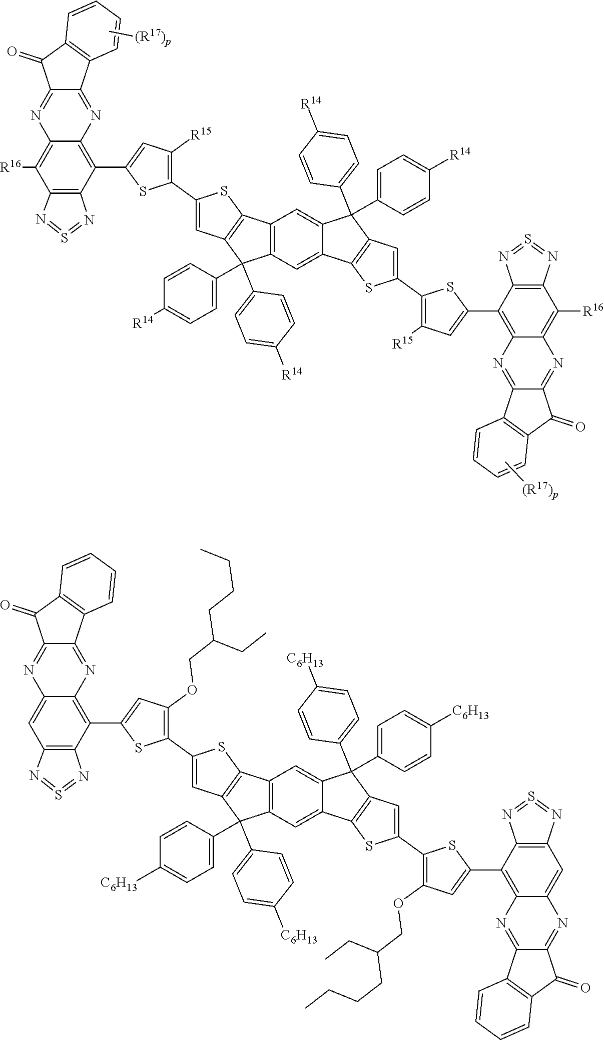

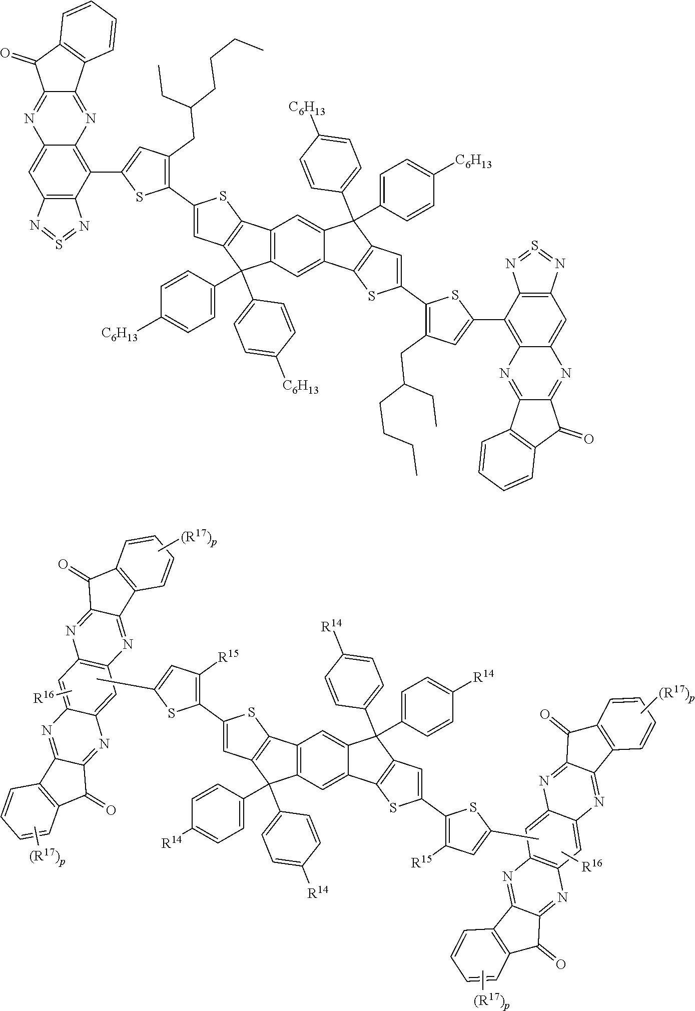

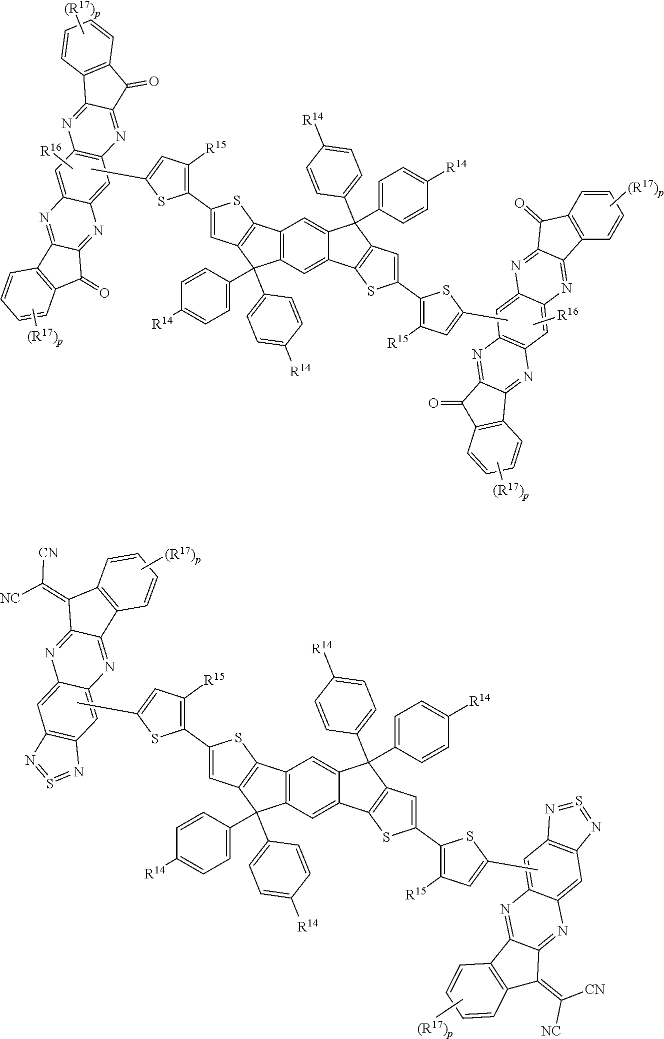

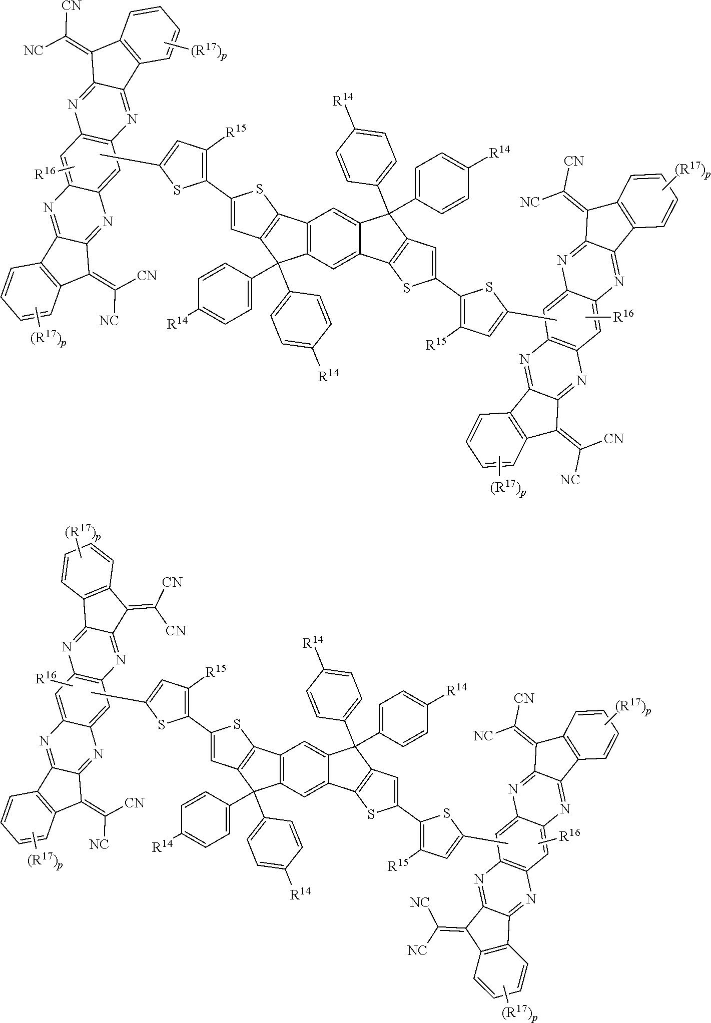

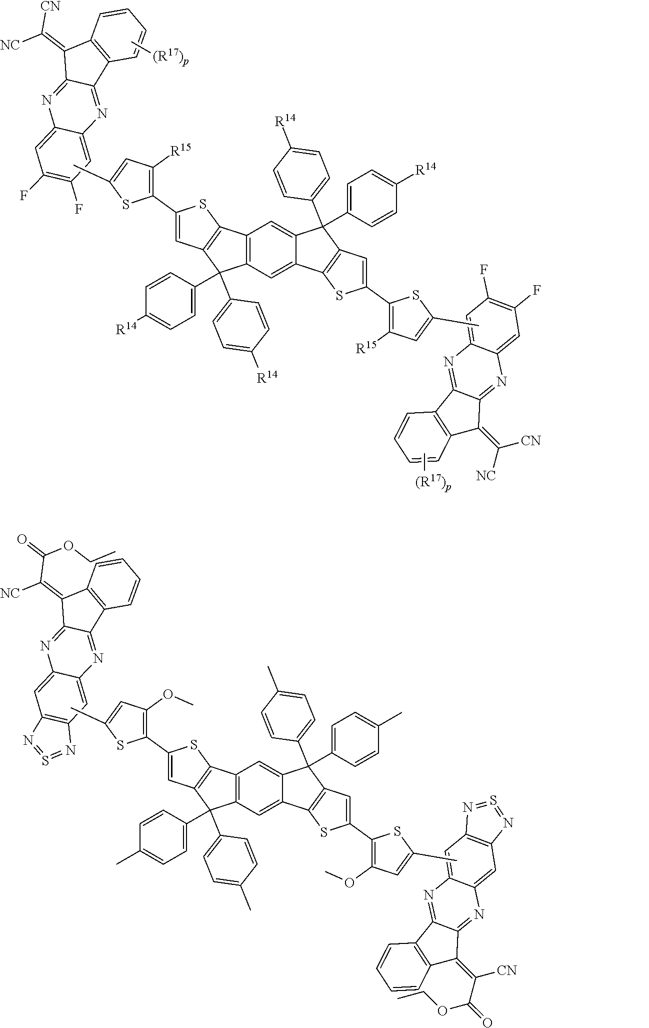

R¹⁴ and R¹⁵ in each occurrence is independently a C₁₋₁₂ alkyl group wherein one or more non-adjacent, non-terminal C atoms may be replaced with O, S, CO or COO.

R¹⁶ in each occurrence is independently selected from H; a C₁₋₁₂ alkyl wherein one or more non-adjacent, non-terminal C atoms may be replaced with O, S, CO or COO; and an aryl group, preferably phenyl, which may be unsubstituted or substituted with one or more substituents, preferably one or more C₁₋₁₂ alkyl or alkoxy substituents.

R¹⁷ in each occurrence is independently selected from F; a C₁₋₁₂ alkyl wherein one or more non-adjacent, non-terminal C atoms may be replaced with O, S, CO or COO; and an aryl group, preferably phenyl, which may be unsubstituted or substituted with one or more substituents, preferably one or more C₁₋₁₂ alkyl or alkoxy substituents.

Non-fullerene acceptors which do not contain a unit of formula (I) are described in, for example, Cheng et. al., “Next-generation organic photovoltaics based on non-fullerene acceptors”, Nature Photonics volume 12, pages 131-142 (2018), the contents of which are incorporated herein by reference, and which include, without limitation, PDI, ITIC, ITIC, IEICO and derivatives thereof, e.g. fluorinated derivatives thereof such as ITIC-4F and IEICO-4F.

Exemplary fullerene electron acceptor materials are C₆₀, C₇₀, C₇₆, C₇₈ and C₈₄ fullerenes or a derivative thereof, including, without limitation, PCBM-type fullerene derivatives including phenyl-C₆₁-butyric acid methyl ester (C₆₀PCBM), TCBM-type fullerene derivatives (e.g. tolyl-C₆₁-butyric acid methyl ester (C₆₀TCBM)), ThCBM-type fullerene derivatives (e.g. thienyl-C₆₁-butyric acid methyl ester (C₆₀ThCBM); and optionally substituted fullerenes in which a C═C bond is replaced with two C═O bonds and/or a C atom is replaced with N, for example as in KLOC-6.

Fullerene derivatives may have formula (III):

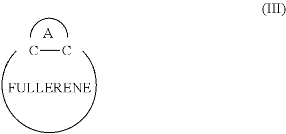

wherein A, together with the C—C group of the fullerene, forms a monocyclic or fused ring group which may be unsubstituted or substituted with one or more substituents.

Exemplary fullerene derivatives include formulae (IIIa), (IIIb) and (IIIc):

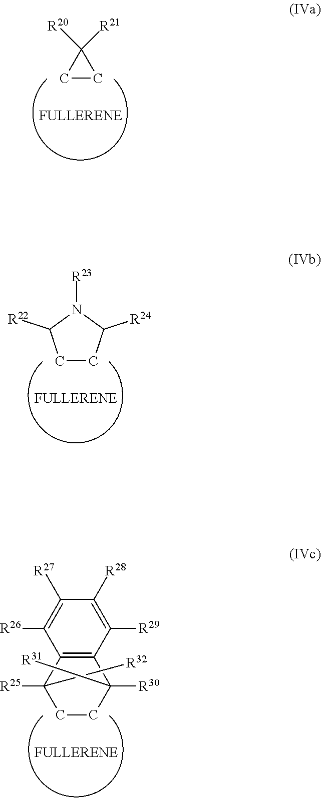

wherein R²⁰-R³² are each independently H or a substituent.

Substituents R²⁰-R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent, non-terminal C atoms may be replaced with O, S, CO or COO and one or more H atoms may be replaced with F.

Substituents of aryl or heteroaryl, where present, are optionally selected from C₁₋₁₂ alkyl wherein one or more non-adjacent, non-terminal C atoms may be replaced with O, S, CO or COO and one or more H atoms may be replaced with F.

### Formulations

The bulk heterojunction layer may be formed by any process including, without limitation, thermal evaporation and solution deposition methods.

Preferably, the bulk heterojunction layer is formed by depositing a formulation comprising the electron donor material(s), the electron acceptor material(s) and any other components of the bulk heterojunction layer dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron acceptor, the electron donor and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

### Organic Electronic Device

A polymer or composition as described herein may be provided as an active layer of an organic electronic device. In a preferred embodiment, a bulk heterojunction layer of an organic photoresponsive device, more preferably an organic photodetector, comprises a composition as described herein.

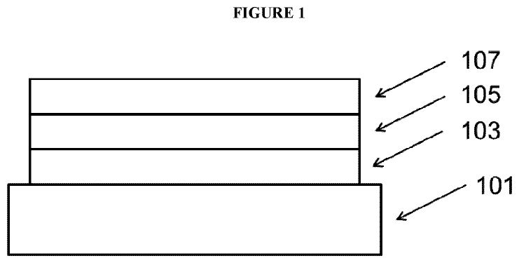

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

At least one of the anode and cathode is transparent so that light incident on the device may reach the bulk heterojunction layer. In some embodiments, both of the anode and cathode are transparent.

Each transparent electrode preferably has a transmittance of at least 70%, optionally at least 80%, to wavelengths in the range of 750-1000 nm or 1300-1400 nm. The transmittance may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer. In some embodiments, a work function modification layer is disposed between the bulk heterojunction layer and the anode, and/or between the bulk heterojunction layer and the cathode.

The area of the OPD may be less than about 3 cm², less than about 2 cm², less than about 1 cm², less than about 0.75 cm², less than about 0.5 cm² or less than about 0.25 cm². Optionally, each OPD may be part of an OPD array wherein each OPD is a pixel of the array having an area as described herein, optionally an area of less than 1 mm², optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer contains a polymer as described herein and an electron acceptor material. The bulk heterojunction layer may consist of these materials or may comprise one or more further materials, for example one or more further electron donor materials and/or one or more further electron acceptor materials.

### Applications

A circuit may comprise the OPD connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 900 nm, optionally in the range of 900-1000 nm. In some embodiments, the light source has a peak wavelength greater than 1000 nm, optionally greater than 1100 nm, optionally greater than 1250 nm, optionally in the range of 1300-1400 nm.

The present inventors have found that a material comprising an electron-accepting unit of formula (I) may be used for the detection of light at longer wavelengths, particularly >1250 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g. due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### EXAMPLES

### Intermediate Compound Example 1

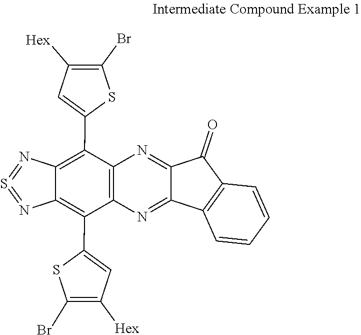

Intermediate Compound Example 1 was prepared following the reported procedure in Macromolecules 2019, 52, p 6149-6159.

### Intermediate Compound Example 2

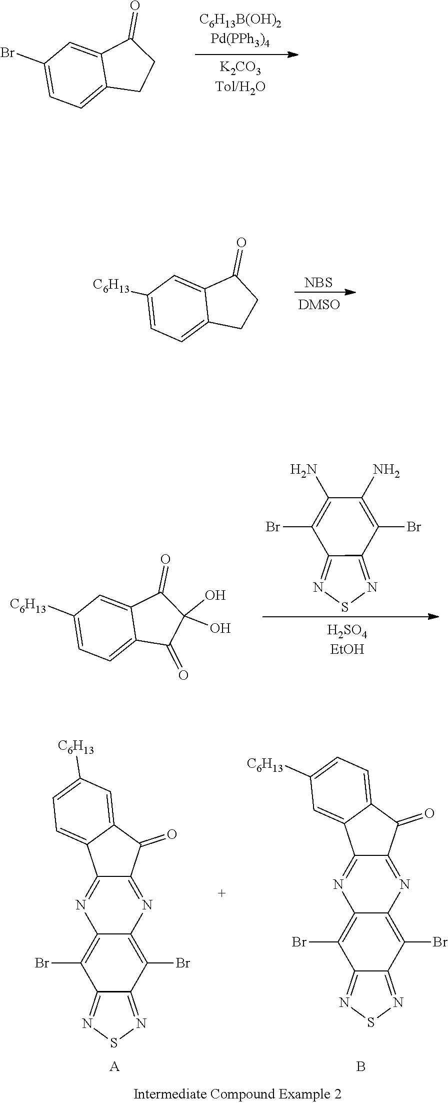

A solution of K₂CO₃ (23.31 g, 169 mmol) in water (70 ml) was added to a degassed solution of 6-bromoindanone (7.12 g, 33.73 mmol), hexyl-boronic acid (8.77 g, 67.47 mmol), Pd(PPh₃)₄ (3.40 g, 3.37 mmol) and toluene. After refluxing overnight, the reaction mixture was cooed to room temperature, separated and the aqueous phase washed with toluene (50 ml). The combined organic layers were dried, evaporated and purified via column chromatography (heptane/ethyl acetate) to give 6-hexyl1-indanone (3.38 g) as an oil.

A solution of 6-hexyl-1-indanone (3.38 g, 15.62 mmol) and N-bromosuccinimide (5.7 g, 32.03 mmol) in DMSO and heated at 80° C. for 6 hrs. The cooled reaction mixture was poured into water (200 ml) and extracted with ethyl acetate, dried, evaporated and purified via recrystallization from heptane/ethyl acetate to give 6-hexyl ninhydrin (1.34 g).

Concentrated sulfuric acid (5 drops) was added to a solution of 6-hexyl-ninhydrin (1.0 g. 3.09 mmol) and 4,7-dibromo-5,6-diamino-2,1,3-benzothiadiazole (1.21 g, 4.63 mmol, prepared as described in Bioconjugate Chemistry, 2016, 27(7), p 1614-1623) in ethanol (35 ml). After heating at 80° C. overnight, the reaction mixture was cooled to room temperature, the solid filtered and washed with water, ethanol, methanol and recrystallized (chloroform/methanol) to give Intermediate Compound Example 2 (1.13 g) as a mixture of 2 isomers.

Isomer A: ¹H NMR (400 MHz, CDCl₃), δ [ppm]: 8.27 (d, 1H, 7.0 Hz); 7.89 (s, 1H); 7.72 (d, 1H, 7.2 Hz); 2.81 (t, 2H, 7.8 Hz), 1.71-1.78 (m, 2H); 1.3-1.4 (m, 6H); 1.72 (m, 3H)

Isomer B: ¹H NMR (400 MHz, CDCl₃), δ [ppm]: 8.17 (s, 1H); 7.99 (d, 1H, 7.8 Hz); 7.60 (d, 1H, 7.9 Hz); 2.86 (t, 2H, 8.0 Hz); 1.71-1.78 (m, 2H), 1.3-1.4 (m, 6H); 1.72 (m, 3H)

### Intermediate Compound Example 3

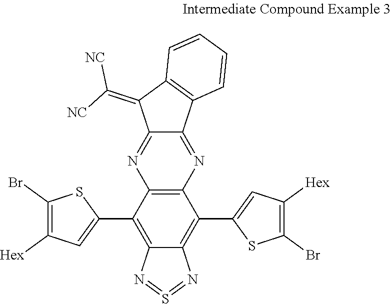

Intermediate Compound Example 3 was prepared following the reported procedure in Macromolecules 2019, 52, p 6149-6159.

### Intermediate Compound Example 4

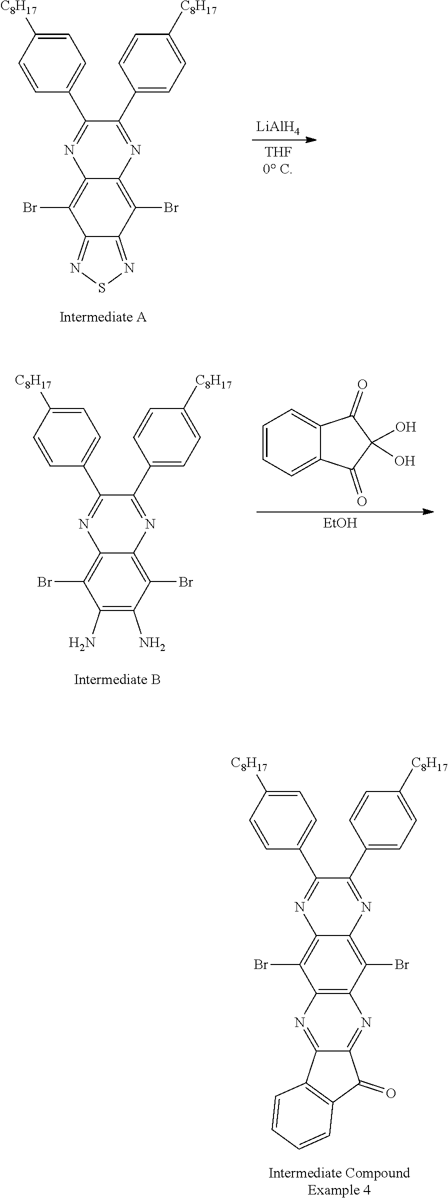

A solution of Intermediate A (6.85 g, 9.48 mmol, prepared as described in Bioconjugate Chemistry, 2016, 27(7), p 1614-1623) in THF (257 ml) of THF was cooled to 0° C. LiAlH₄ (37.92 ml, 37.92 mmol, 1M in THF) was added dropwise. After 30 minutes the reaction mixture was quenched with water, evaporated dissolved in ethyl acetate and filtered. Precipitation from heptane, gave Intermediate B (4.24 g) as a yellow solid.

A solution of Intermediate B (4.24 g, 5.87 mmol) and ninhydrin (4.18 g, 23.47 mmol) in ethanol (20 ml) was heated at reflux overnight. The reaction mixture was cooled, and the resulting orange precipitate was filtered, washed with ethanol and recrystallized (isopropanol/chloroform) to give Intermediate Compound Example 4 (3.70 g).

¹H NMR (400 MHz, CDCl₃), δ [ppm]: 8.38 (d, 1H, 7.6 Hz); 8.05 (d, 1H, 7.8 Hz); 7.88 (t, 1H, 7.5 Hz); 7.69-7.74 (m, 5H); 7.20-7.22 (m, 4H); 2.64-2.67 (m, 4H); 1.61-1.65 (m, 4H); 1.27-1.32 (m, 20H); 0.88 (t, 6H, 7.1 Hz).

### Intermediate Compound 5

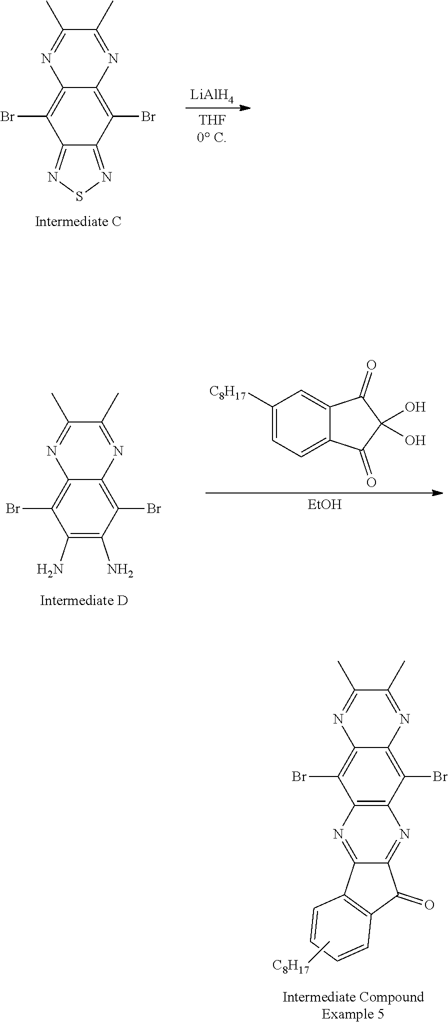

Intermediate C (4.80 g; 12.83, 1 eq) was dissolved in 120 ml of dry THF under nitrogen atmosphere. This solution was cooled to −20° C. and LiAlH4 (1M in THF; 12.83 mL) was added dropwise withing 10 minutes. Slowly brought to room temperature and stirred for 3.5 h, cooled to 0° C. and quenched with 5 mL of water. Solvent was removed and the residue was extracted with ethyl acetate, filtrated, diluted with heptane and evaporated. Recrystallized from ethyl acetate/methanol mixture to give 2.63 g of intermediate D.

Ninhydrin-C8 (2.77 g; 9.28 mmol) dissolved in warm ethanol (75 ml) and Intermediate D was added, heated to reflux overnight. Brought to room temperature, filtered, washed with methanol and heptane. Purified via column chromatography (reverse phase-C18; MeCN/THF; 4%-36%), filtered from methanol to give intermediate compound 5 0.54 g as a mixture of 2 isomers (ratio 1:3).

¹H NMR (400 MHz, CDCl₃), δ [ppm]: 8.23 (d, 1H); 8.14 (s, 1H); 7.94 (d, 1H); 7.85 (s, 1H); 7.67 (d, 1H); 7.53 (d, 1H); 2.92 (s, 6H); 2.85 (t, 2H); 2.78 (t, 2H); 1.70-1.78 (m, 2H); 1.29-1.56 (m, 10H), 0.88 (t, 3H).

Intermediate Compound Examples 1-5 may be reacted to form polymeric or non-polymeric materials comprising an electron-accepting unit derived from these compounds and an electron-donating unit.

### Intermediate Compound 6

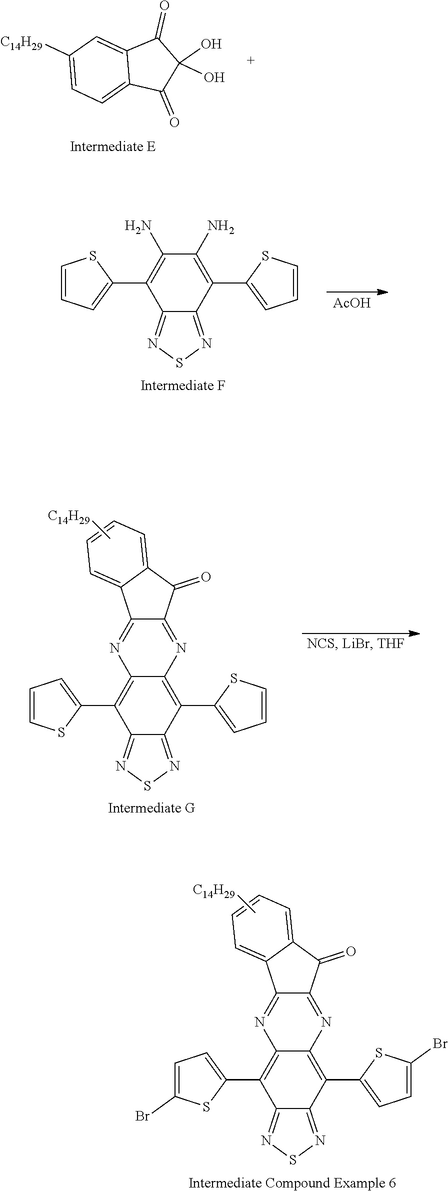

Intermediate F (5.25 g, 15.9 mmol) was added to 3-neck flask and slurried in 200 mL of acetic acid and heated until dissolved at 120° C. Then Intermediate E (5 g, 15.9 mmol) was added in small portions over 20 minutes and then refluxed for 1 hour. The reaction mixture was cooled in ice, diluted with methanol and filtered. Recrystallisation from a THF/methanol mixture gave 6.87 g of Intermediate G as a mixture of isomers.

¹H NMR (400 MHz, CDCl₃, δ ppm): 8.87 (m, 2H); 8.84 (m, 2H); 8.02 (d, 1H, 7.7 Hz); 7.90 (s, 1H); 7.88 (d, 1H, 7.6 Hz); 7.77 (s, 1H); 7.70-7.68 (m, 2H); 7.61-7.60 (m, 2H); 7.54 (d, 1H, 5.0 Hz); 7.48 (d, 1H, 7.4 Hz); 7.25-7.23 (m, 2H); 7.21-7.19 (m, 1H); 7.17-7.16 (m, 1H); 2.84 (t, 2H, 7.81 Hz); 2.79 (t, 2H, 7.81 Hz); 1.86-1.71 (m, 4H), 1.49-1.44 (m, 4H); 1.42-1.25 (m, 44 H); 0.89-0.84 (m, 6H).

To Intermediate G (2.25 g, 3.42 mmol) in 3 neck flask was added LiBr (0.563 g, 6.49 mmol) in dry THF (100 mL) under nitrogen atmosphere. The mixture was cooled to −40° C. and N-chlorosuccinimide (0.866 g, 6.49 mmol) dissolved in dry THF (15 mL) was added slowly and stirred at this temperature for 1 hour. Additional portions of N-chlorosuccinimide (0.092 g) and LiBr (0.060 g) were added and stirred for additional 2 hours. The reaction mixture was brought to room temperature and stirred for 1 hour. Water was added and the mixture was extracted with DCM, washed with water, and dried over MgSO₄. Recrystallisation from heptane/methanol gave 0.84 g of Intermediate Compound Example 6 as a mixture of isomers.

¹H NMR (400 MHz, CDCl₃, δ ppm): 8.16 (d, 1H, 4.0 Hz); 8.11 (d, 1H, 3.75 Hz); 7.97 (d, 1H, 3.75 Hz); 7.90 (d, 1H, 4.0 Hz); 7.46 (d, 1H, 7.15 Hz); 7.39 (s, 1H); 7.32 (d, 1H, 7.3 Hz); 7.27 (m, 1H); 7.19-7.16 (m, 1H); 7.07 (s, 1H); 6.73 (d, 1H, 3.2 Hz); 6.71 (d, 1H, 4.0 Hz); 6.38 (d, 1H, 3.6 Hz); 6.27 (d, 1H, 3.6 Hz); 2.71-2.66 (m, 2H); 1.71-1.66 (m, 2H); 1.42-1.25 (m, 22H); 0.91-0.88 (m, 3H).

Polymer Example 1-6, may be formed by Suzuki-Miyaura polymerisation of Intermediate Compound Example 1-6 respectively with a monomer for forming an electron-donating repeat unit, for example as disclosed in U.S. Pat. No. 9,512,149, the contents of which are incorporated herein by reference.

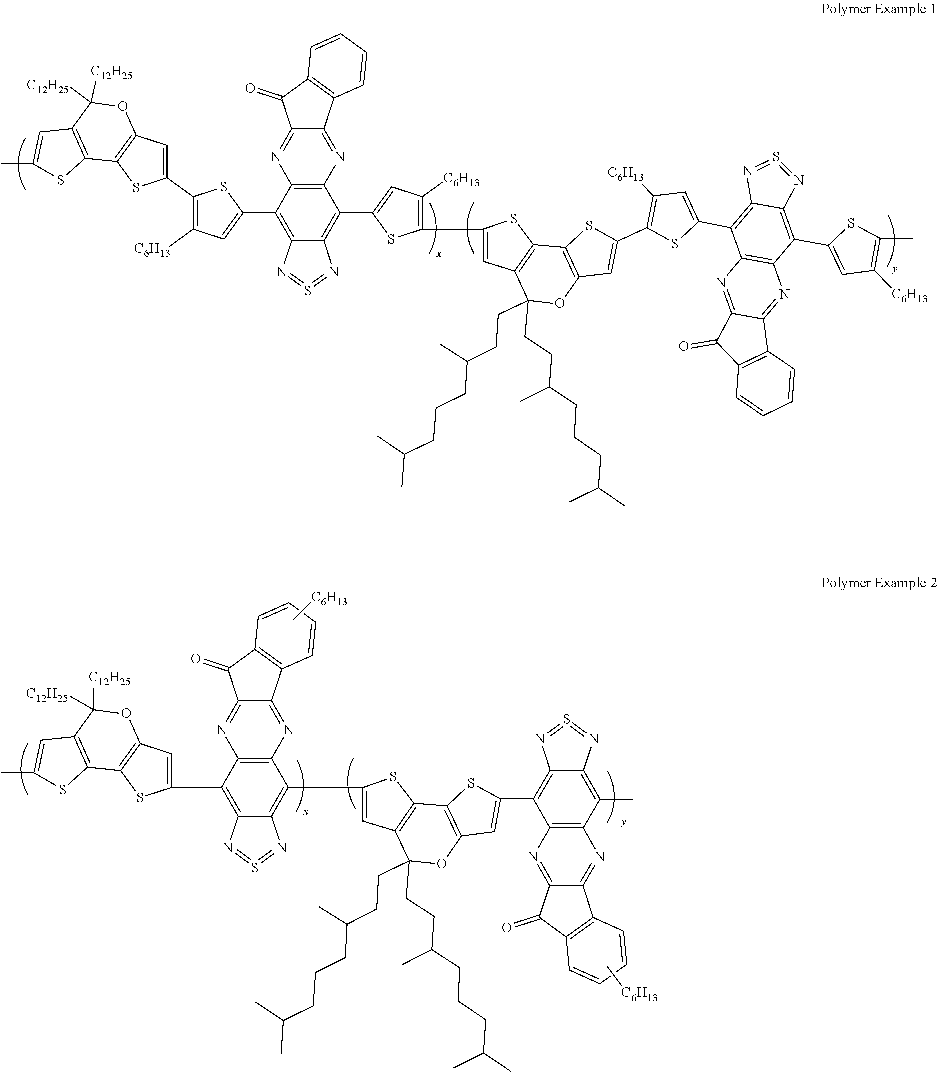

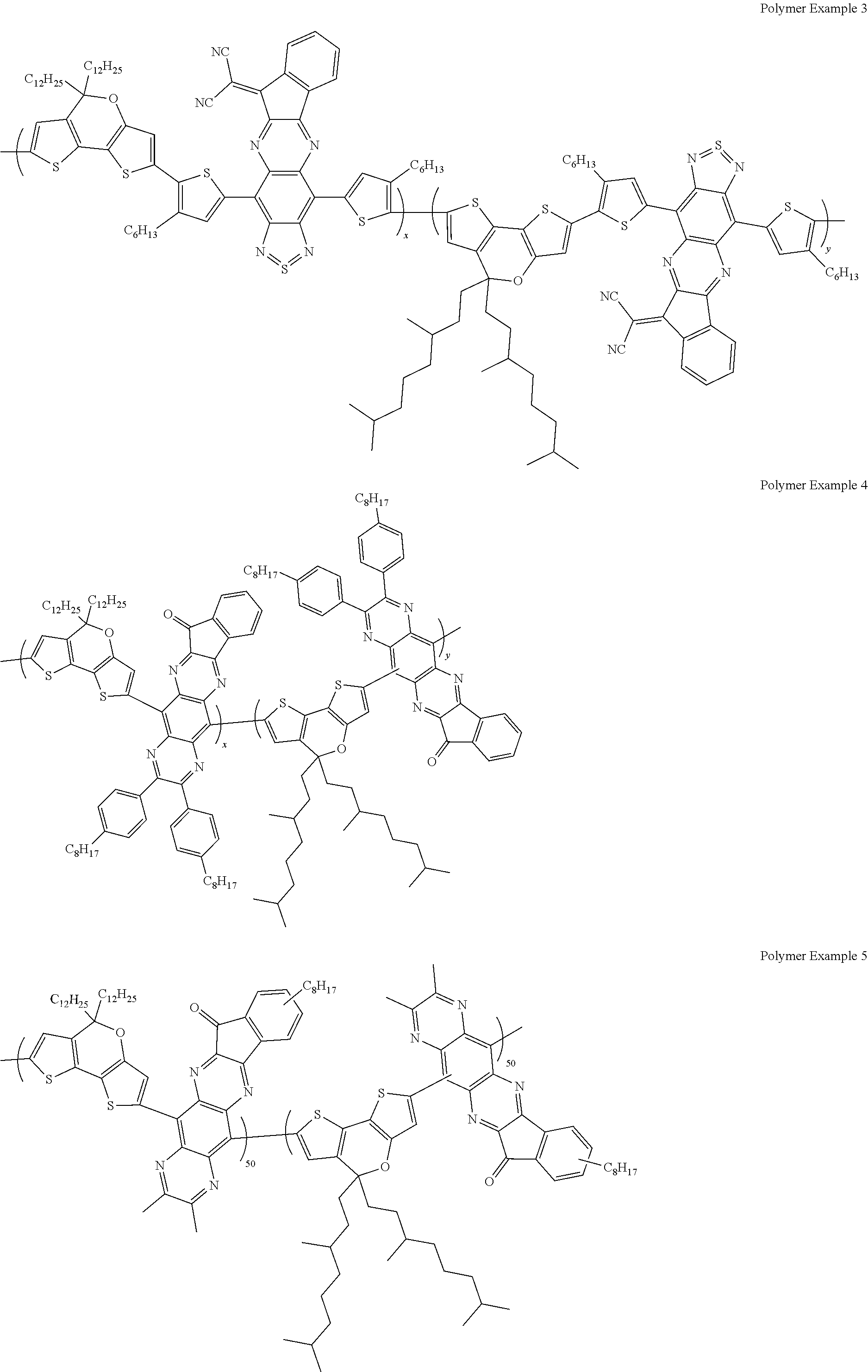

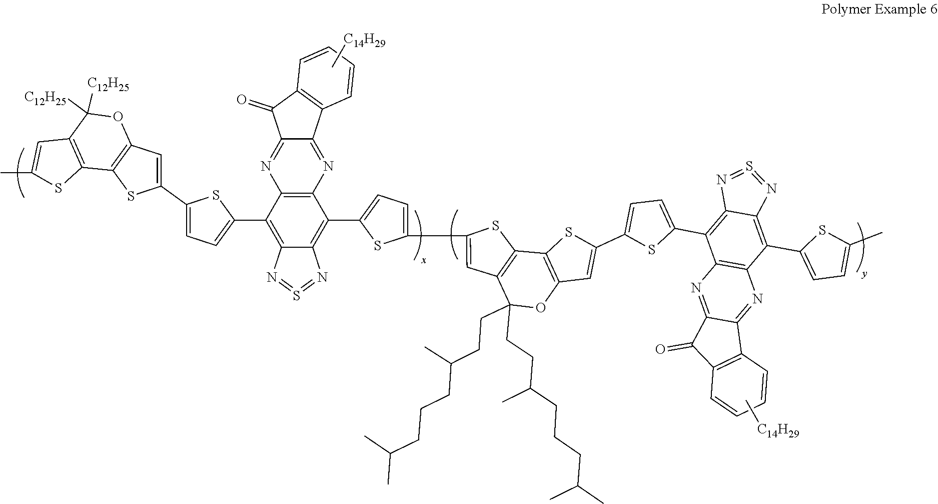

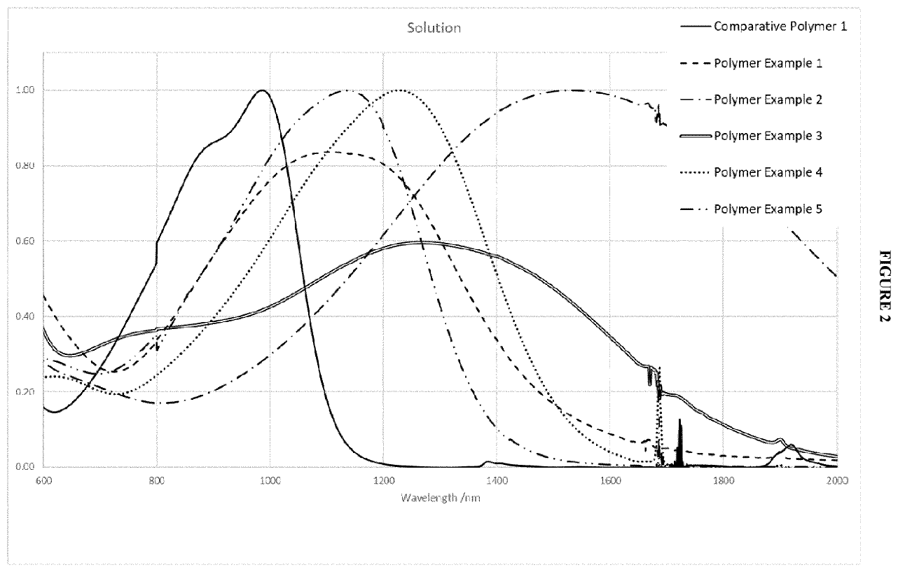

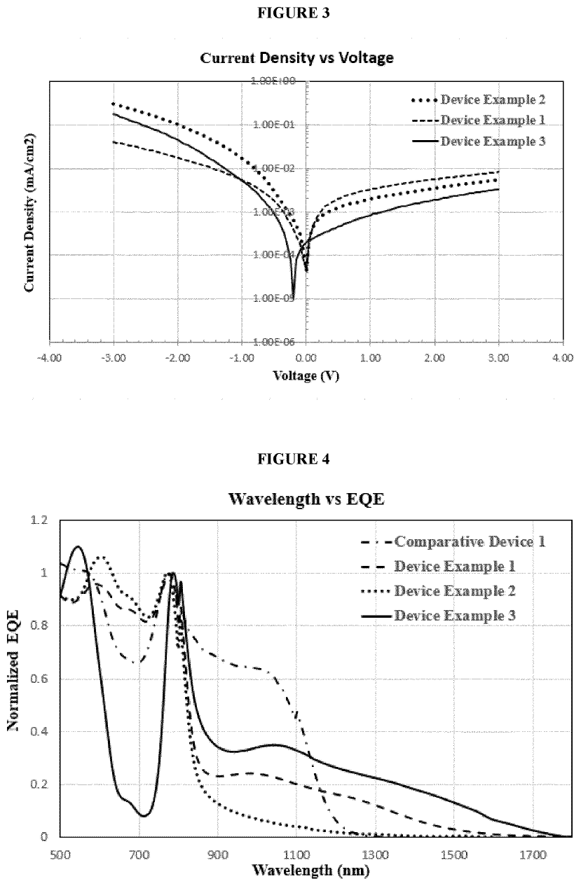

With reference to the absorption spectra of FIG. 2, recorded in toluene solution, Polymer Examples 1-5 show stronger absorption than Comparative Polymer 1 at wavelengths above about 1100 nm.

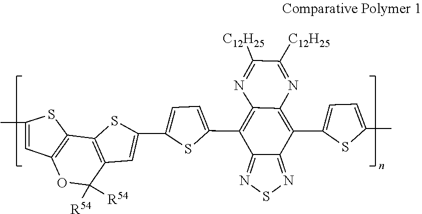

In Comparative Polymer 1, R⁵⁴ is 3,7-dimethyloctyl for 50% of n and is C₁₂H₂₅ for the other 50%.

Table A contains HOMO and LUMO values as measured by SWV for Polymer Examples 1 to 4 and Comparative Polymer 1.

| Polymer | HOMO (eV) | LUMO (eV) | Band gap (eV) |
|---|---|---|---|
| Comparative Polymer 1 | −5.05 | −3.45 | 1.6 |
| Polymer Example 1 | −5.07 | −3.77 | 1.3 |
| Polymer Example 2 | −4.91 | −3.92 | 0.99 |
| Polymer Example 3 | −5.10 | −3.95 | 1.15 |
| Polymer Example 4 | −4.84 | −3.74 | 1.1 |
| Polymer Example 5 | −4.88 | −3.63 | 1.25 |
| Polymer Example 6 | −5.06 | −3.75 | 1.31 |

### Intermediate Compound Example 7

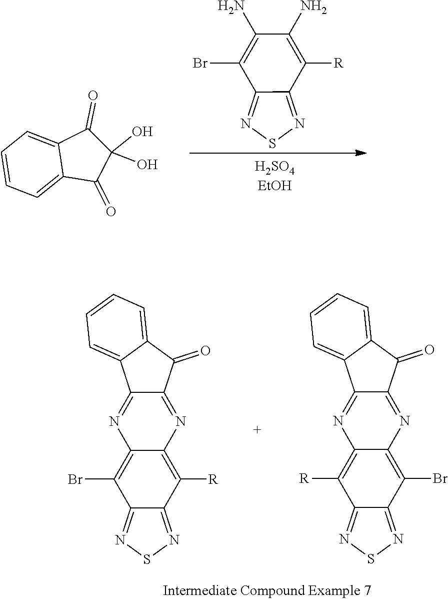

Intermediate Compound Example 7, wherein R represents alkyl or substituted phenyl, e.g. alkyl-substituted phenyl, may be prepared according to the method described for Intermediate Compound Example 2.

A non-polymeric material comprising an electron-accepting repeat unit formed by reaction of Intermediate Compound Example 8 may be formed, for example as shown below.

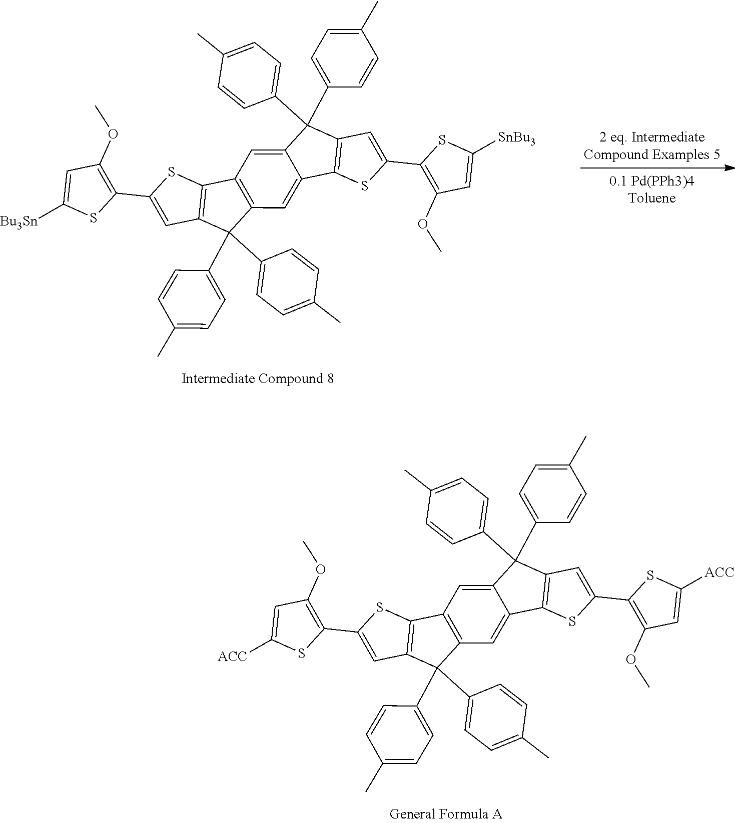

Intermediate Compound Example 8 may be prepared via standard lithiation and stannylation methods, analogous to that disclosed in for example US20190181348.

### General Formula A

Intermediate Compound Example 8 (1 equiv.), Intermediate Compound Example 5 (2.2 equiv.) and Pd(PPh₃)₄ (0.1 equiv.) are dissolved in toluene under nitrogen and heated to 100° C. for 48 hours. The mixture is allowed to cool, poured into dilute aqueous KF, extracted with DCM and purified. This is analogous to that disclosed in for example CN104557968.

### Quantum Chemical Modelling Example 1

All modelling as described in these examples was performed using Gaussian09 software available from Gaussian using Gaussian09 with B3LYP (functional).

It will be understood that modelling data is not measured in the same way as SWV as described herein.

HOMO and LUMO levels for acceptor (ACC) of model compounds of General Formula 1 were modelled:

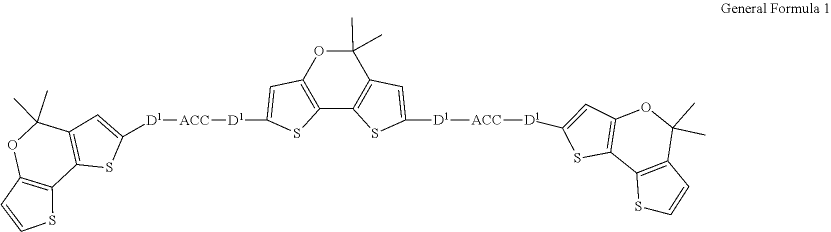

TABLE 1

| | | HOMO | LUMO | E_{g} | Abs |
|---|---|---|---|---|---|
| Entry | D¹-ACC-D¹ | (eV) | (eV) | (eV) | (nm) |
| Comparative Example 1-1 | 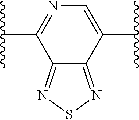 [SMILES: *c1cnc(*)c2nsnc12] | −4.60 | −3.06 | 1.54 | 803 |
| Model Example 1-1 | 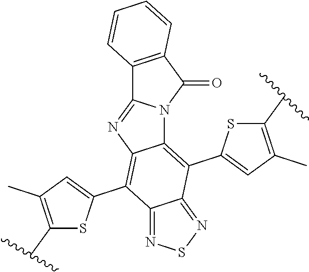 | −4.37 | −2.90 | 1.48 | 839 |
| Model Example 1-2 | 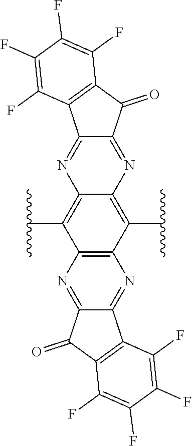 [SMILES: *c1c2nc3c(=O)c4c(F)c(F)c(F)c(F)c4c3nc2c(*)c2nc3c(=O)c4c(F)c(F)c(F)c(F)c4c3nc12] | −4.59 | −3.72 | 0.87 | 1430 |
| Model Example 1-3 | 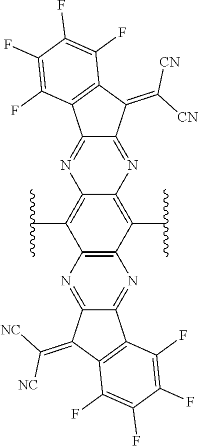 [SMILES: *c1c2nc3c(nc2c(*)c2nc4c(nc12)-c1c(F)c(F)c(F)c(F)c1C4=C(C#N)C#N)-c1c(F)c(F)c(F)c(F)c1C3=C(C#N)C#N] | −4.82 | −4.10 | 0.72 | 1724 |
| Model Example 1-4 | 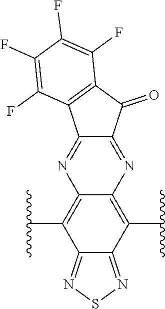 [SMILES: *c1c2nsnc2c(*)c2nc3c(nc12)C(=O)c1c(F)c(F)c(F)c(F)c1-3] | −4.54 | −3.68 | 0.87 | 1433 |
| Model Example 1-5 | 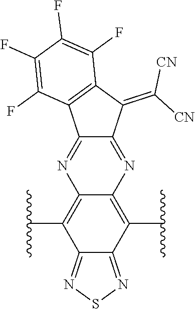 [SMILES: *c1c2nsnc2c(*)c2nc3c(nc12)C(=C(C#N)C#N)c1c(F)c(F)c(F)c(F)c1-3] | −4.67 | −3.88 | 0.79 | 1569 |
| Model Example 1-6 | 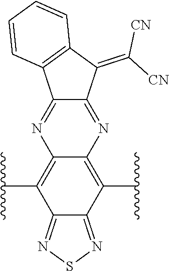 [SMILES: *c1c2nsnc2c(*)c2nc3c(nc12)C(=C(C#N)C#N)c1ccccc1-3] | −4.58 | −3.71 | 0.86 | 1437 |
| Model Example 1-7 | 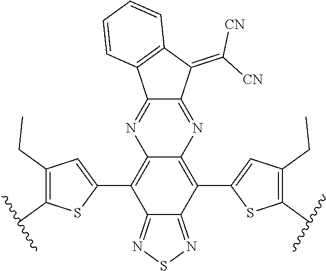 [SMILES: *c1sc(-c2c3nsnc3c(-c3cc(CC)c(*)s3)c3nc4c(nc23)C(=C(C#N)C#N)c2ccccc2-4)cc1CC] | −4.60 | −3.60 | 1.00 | 1237 |
| Model Example 1-8 | 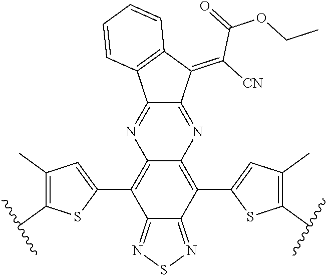 [SMILES: *c1sc(-c2c3nsnc3c(-c3cc(C)c(*)s3)c3nc4c(nc23)/C(=C(\C#N)C(=O)OCC)c2ccccc2-4)cc1C] | −4.34 | −3.43 | 0.91 | 1365 |
| Model Example 1-9 | 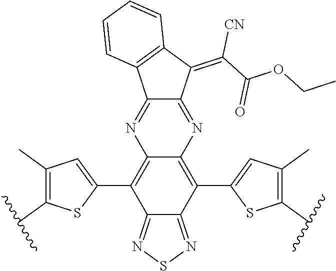 [SMILES: *c1sc(-c2c3nsnc3c(-c3cc(C)c(*)s3)c3nc4c(nc23)/C(=C(/C#N)C(=O)OCC)c2ccccc2-4)cc1C] | −4.47 | −3.48 | 0.99 | 1255 |
| Model Example 1-10 | 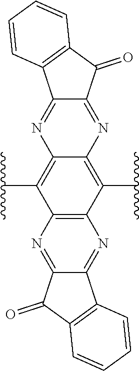 [SMILES: *c1c2nc3c(=O)c4ccccc4c3nc2c(*)c2nc3c(=O)c4ccccc4c3nc12] | −4.44 | −3.40 | 1.04 | 1190 |
| Model Example 1-11 | 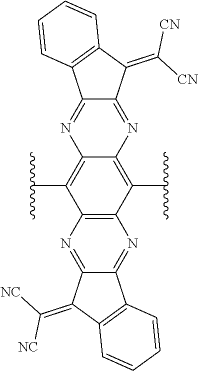 [SMILES: *c1c2nc3c(nc2c(*)c2nc4c(nc12)-c1ccccc1C4=C(C#N)C#N)-c1ccccc1C3=C(C#N)C#N] | −4.65 | −3.88 | 0.77 | 1608 |
| Model Example 1-12 | 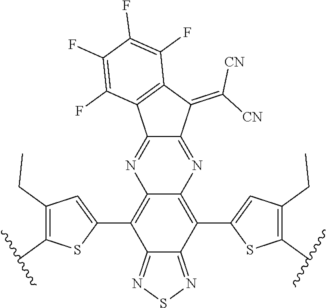 [SMILES: *c1sc(-c2c3nsnc3c(-c3cc(CC)c(*)s3)c3nc4c(nc23)C(=C(C#N)C#N)c2c(F)c(F)c(F)c(F)c2-4)cc1CC] | −4.64 | −3.77 | 0.87 | 1419 |
| Model Example 1-13 | 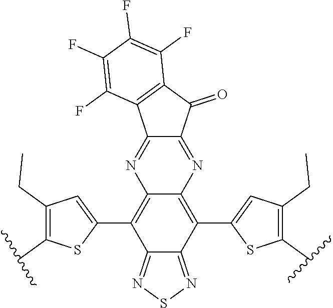 [SMILES: *c1sc(-c2c3nsnc3c(-c3cc(CC)c(*)s3)c3nc4c(nc23)C(=O)c2c(F)c(F)c(F)c(F)c2-4)cc1CC] | −4.53 | −3.53 | 1.00 | 1242 |
| Model Example 1-14 | 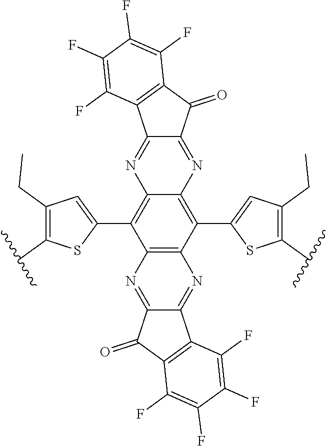 [SMILES: *c1sc(-c2c3nc4c(=O)c5c(F)c(F)c(F)c(F)c5c4nc3c(-c3cc(CC)c(*)s3)c3nc4c(=O)c5c(F)c(F)c(F)c(F)c5c4nc23)cc1CC] | −4.51 | −3.58 | 0.93 | 1340 |
| Model Example 1-15 | 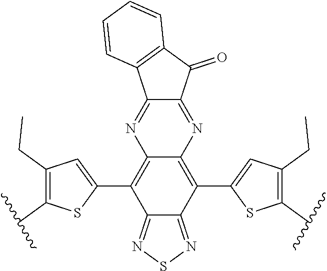 [SMILES: *c1sc(-c2c3nsnc3c(-c3cc(CC)c(*)s3)c3nc4c(nc23)C(=O)c2ccccc2-4)cc1CC] | −4.45 | −3.35 | 1.10 | 1128 |
| Model Example 1-16 | 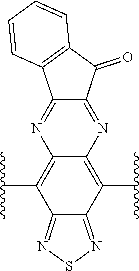 [SMILES: *c1c2nsnc2c(*)c2nc3c(nc12)C(=O)c1ccccc1-3] | −4.43 | −3.48 | 0.95 | 1306 |
| Model Example 1-17 | 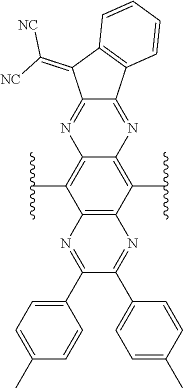 [SMILES: *c1c2nc3c(nc2c(*)c2nc(-c4ccc(C)cc4)c(-c4ccc(C)cc4)nc12)-c1ccccc1C3=C(C#N)C#N] | −4.47 | −3.43 | 1.04 | 1194 |
| Model Example 1-18 | 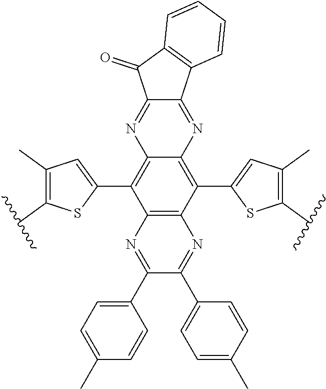 [SMILES: *c1sc(-c2c3nc4c(nc3c(-c3cc(C)c(*)s3)c3nc(-c5ccc(C)cc5)c(-c5ccc(C)cc5)nc23)-c2ccccc2C4=O)cc1C] | −4.28 | −3.10 | 1.18 | 1051 |
| Model Example 1-19 | 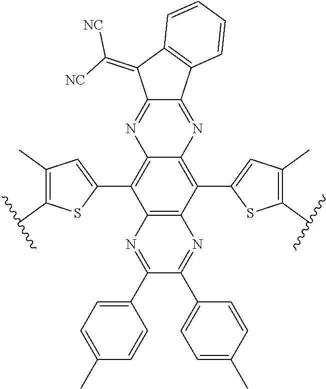 [SMILES: *c1sc(-c2c3nc4c(nc3c(-c3cc(C)c(*)s3)c3nc(-c5ccc(C)cc5)c(-c5ccc(C)cc5)nc23)-c2ccccc2C4=C(C#N)C#N)cc1C] | −4.41 | −3.38 | 1.03 | 1201 |
| Model Example 1-20 | 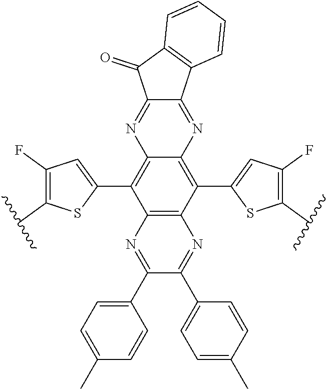 [SMILES: *c1sc(-c2c3nc4c(nc3c(-c3cc(F)c(*)s3)c3nc(-c5ccc(C)cc5)c(-c5ccc(C)cc5)nc23)-c2ccccc2C4=O)cc1F] | −4.39 | −3.27 | 1.12 | 1107 |
| Model Example 1-21 | 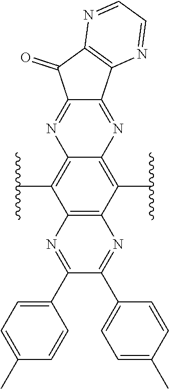 [SMILES: *c1c2nc3c(nc2c(*)c2nc(-c4ccc(C)cc4)c(-c4ccc(C)cc4)nc12)-c1nccnc1C3=O] | −4.39 | −3.33 | 1.06 | 1172 |
| Model Example 1-22 | 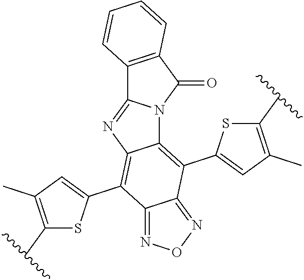 | −4.52 | −2.92 | 1.60 | 773 |
| Model Example 1-23 | 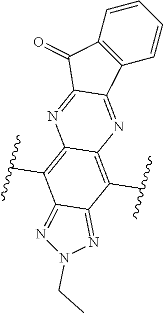 [SMILES: *c1c2nc3c(nc2c(*)c2nn(CC)nc12)-c1ccccc1C3=O] | −4.34 | −3.06 | 1.28 | 966 |
| Model Example 1-24 | 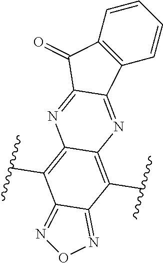 [SMILES: *c1c2nonc2c(*)c2nc3c(nc12)C(=O)c1ccccc1-3] | −4.54 | −3.60 | 0.93 | 1320 |
| Model Example 1-25 | 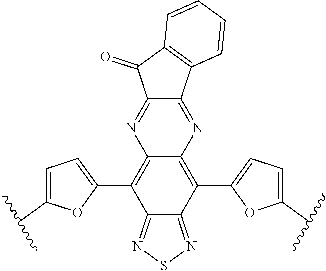 [SMILES: *c1ccc(-c2c3nsnc3c(-c3ccc(*)o3)c3nc4c(nc23)C(=O)c2ccccc2-4)o1] | −4.35 | −3.30 | 1.05 | 1181 |
| Model Example 1-26 | 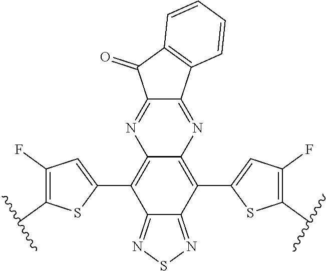 [SMILES: *c1sc(-c2c3nsnc3c(-c3cc(F)c(*)s3)c3nc4c(nc23)C(=O)c2ccccc2-4)cc1F] | −4.45 | −3.56 | 0.90 | 1384 |
| Model Example 1-27 | 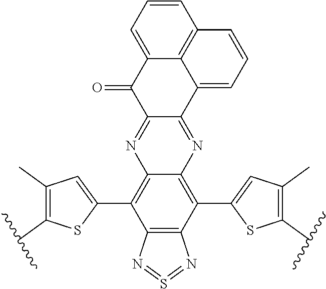 [SMILES: *c1sc(-c2c3c(c(-c4cc(C)c(*)s4)c4nc5c(nc24)C(=O)c2cccc4cccc-5c24)N=S=N3)cc1C] | −4.34 | −3.39 | 0.95 | 1311 |
| Model Example 1-28 | 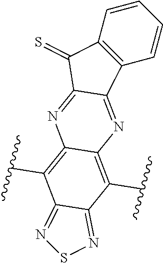 [SMILES: *c1c2nsnc2c(*)c2nc3c(nc12)C(=S)c1ccccc1-3] | −3.59 | −4.43 | 0.84 | 1480 |
| Model Example 1-29 | 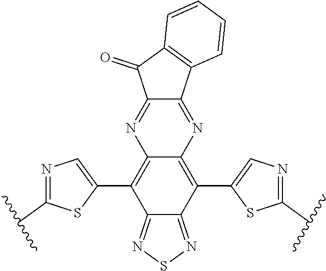 [SMILES: *c1ncc(-c2c3nsnc3c(-c3cnc(*)s3)c3nc4c(nc23)C(=O)c2ccccc2-4)s1] | −4.67 | −3.53 | 1.134 | 1091 |
| Model Example 1-30 | 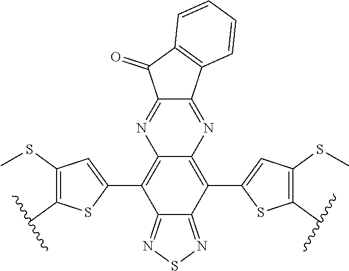 | −4.70 | −3.49 | 1.21 | 1025 |
| Model Example 1-31 | 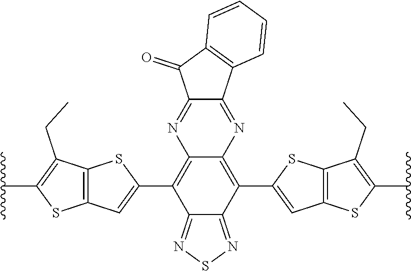 [SMILES: *c1sc2cc(-c3c4nsnc4c(-c4cc5sc(*)c(CC)c5s4)c4nc5c(nc34)C(=O)c3ccccc3-5)sc2c1CC] | −4.60 | −3.43 | 1.18 | 1053 |
| Model Example 1-32 | 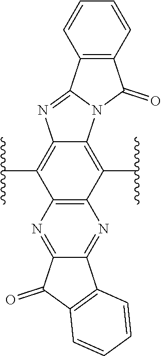 [SMILES: *c1c2nc3c(=O)c4ccccc4c3nc2c(*)c2c1nc1c3ccccc3c(=O)n12] | −4.48 | −2.87 | 1.61 | 770 |
| Model Example 1-33 | 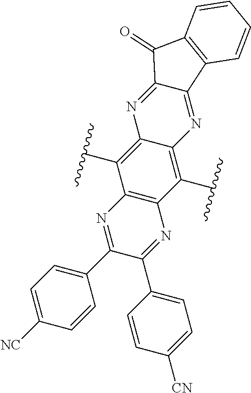 [SMILES: *c1c2nc3c(nc2c(*)c2nc(-c4ccc(C#N)cc4)c(-c4ccc(C#N)cc4)nc12)-c1ccccc1C3=O] | −4.65 | −3.50 | 1.16 | 1073 |
| Model Example 1-34 | 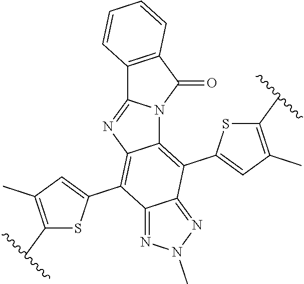 | −4.40 | −2.53 | 1.87 | 662 |
| Model Example 1-35 | 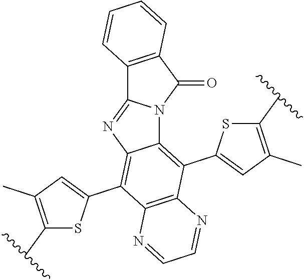 | −4.37 | −2.75 | 1.62 | 767 |
| Model Example 1-36 | 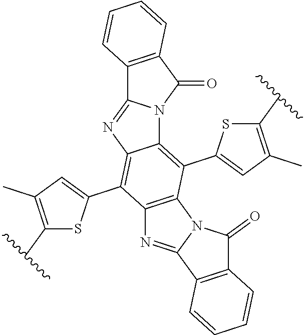 | −4.50 | −2.43 | 2.06 | 601 |
| Model Example 1-37 | 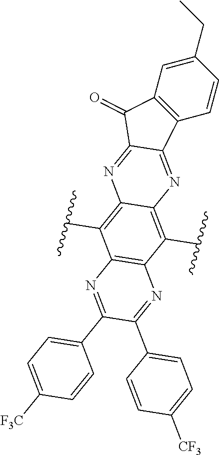 [SMILES: *c1c2nc3c(nc2c(*)c2nc(-c4ccc(C(F)(F)F)cc4)c(-c4ccc(C(F)(F)F)cc4)nc12)-c1ccc(CC)cc1C3=O] | −4.49 | −3.30 | 1.19 | 1045 |
| Model Example 1-38 | 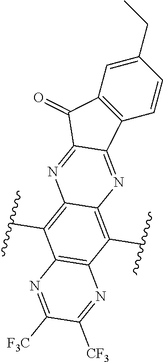 [SMILES: *c1c2nc3c(nc2c(*)c2nc(C(F)(F)F)c(C(F)(F)F)nc12)-c1ccc(CC)cc1C3=O] | −4.55 | −3.55 | 1.00 | 1237 |
| Model Example 1-39 | 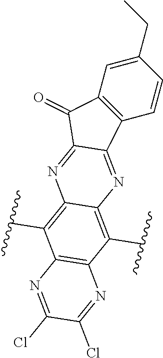 [SMILES: *c1c2nc(Cl)c(Cl)nc2c(*)c2nc3c(nc12)C(=O)c1cc(CC)ccc1-3] | −4.51 | −3.40 | 1.11 | 1119 |
| Model Example 1-40 | 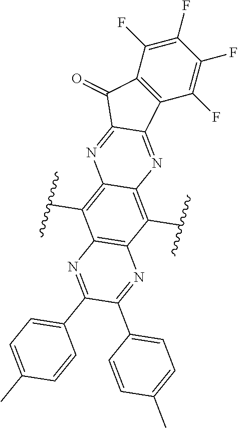 [SMILES: *c1c2nc3c(nc2c(*)c2nc(-c4ccc(C)cc4)c(-c4ccc(C)cc4)nc12)-c1c(F)c(F)c(F)c(F)c1C3=O] | −4.42 | −3.29 | 1.12 | 1104 |
| Model Example 1-41 | 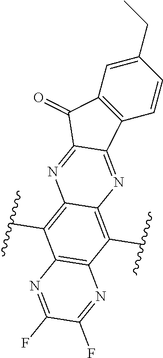 [SMILES: *c1c2nc(F)c(F)nc2c(*)c2nc3c(nc12)C(=O)c1cc(CC)ccc1-3] | −4.50 | −3.29 | 1.20 | 1029 |
| Model Example 1-42 | 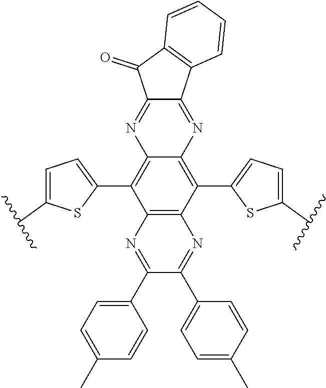 [SMILES: *c1ccc(-c2c3nc4c(nc3c(-c3ccc(*)s3)c3nc(-c5ccc(C)cc5)c(-c5ccc(C)cc5)nc23)-c2ccccc2C4=O)s1] | −4.36 | −3.10 | 1.26 | 985 |

### Quantum Chemical Modelling Example 2

HOMO and LUMO levels for model compounds of General Formula 2 were modelled:

D-D¹-ACC-D¹-D-D¹-ACC-D¹-D General Formula 2

TABLE 2

| | | | HOMO | LUMO | E_{g} | Abs |
|---|---|---|---|---|---|---|
| Name | D | D¹-ACC-D¹ | (eV) | (eV) | (eV) | (nm) |
| Model Example 2-1 | 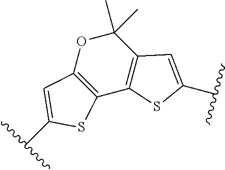 [SMILES: *c1cc2c(s1)-c1sc(*)cc1C(C)(C)O2] | 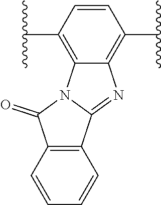 [SMILES: *c1ccc(*)c2c1nc1n2C(=O)c2ccccc2-1] | −4.59 | −2.49 | 2.06 | 603 |
| Comparative Model 2-1 | 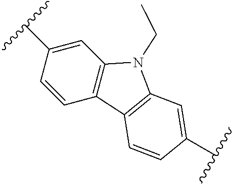 [SMILES: *c1ccc2c3ccc(*)cc3n(CC)c2c1] | 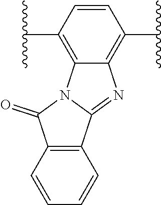 [SMILES: *c1ccc(*)c2c1nc1n2C(=O)c2ccccc2-1] | −5.11 | −2.40 | 2.71 | 458 |
| Comparative Model 2-2 | 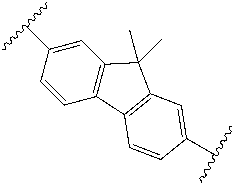 [SMILES: *c1ccc2c(c1)C(C)(C)c1cc(*)ccc1-2] | 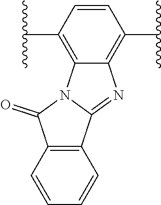 [SMILES: *c1ccc(*)c2c1nc1n2C(=O)c2ccccc2-1] | −5.17 | −2.35 | 2.83 | 438 |
| Model Example 2-2 | 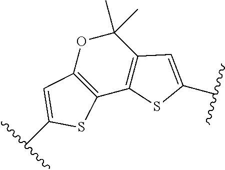 [SMILES: *c1cc2c(s1)-c1sc(*)cc1C(C)(C)O2] | 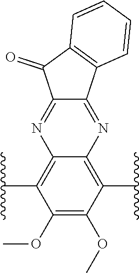 [SMILES: *c1c(OC)c(OC)c(*)c2nc3c(nc12)C(=O)c1ccccc1-3] | −4.49 | −2.74 | 1.73 | 715 |
| Comparative Model 2-3 | 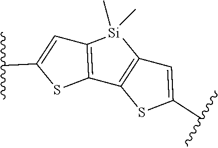 [SMILES: *c1cc2c(s1)-c1sc(*)cc1[Si]2(C)C] | 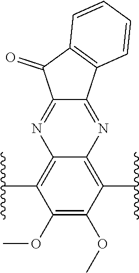 [SMILES: *c1c(OC)c(OC)c(*)c2nc3c(nc12)C(=O)c1ccccc1-3] | −4.61 | −2.70 | 1.91 | 650 |
| Comparative Model 2-4 | 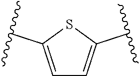 [SMILES: *c1ccc(*)s1] | 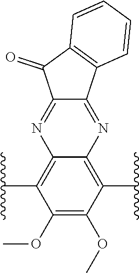 [SMILES: *c1c(OC)c(OC)c(*)c2nc3c(nc12)C(=O)c1ccccc1-3] | −5.01 | −2.71 | 2.31 | 538 |
| Model Example 2-3 | 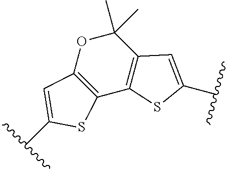 [SMILES: *c1cc2c(s1)-c1sc(*)cc1C(C)(C)O2] | 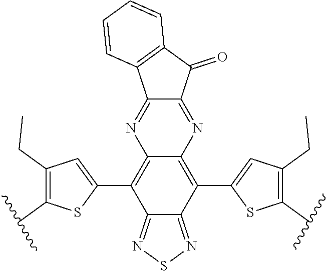 [SMILES: *c1sc(-c2c3nsnc3c(-c3cc(CC)c(*)s3)c3nc4c(nc23)C(=O)c2ccccc2-4)cc1CC] | −4.45 | −3.35 | 1.10 | 1128 |
| Model Example 2-4 | 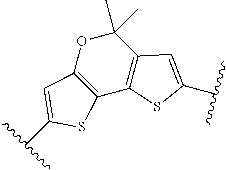 [SMILES: *c1cc2c(s1)-c1sc(*)cc1C(C)(C)O2] | 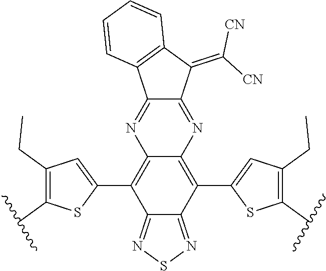 [SMILES: *c1sc(-c2c3nsnc3c(-c3cc(CC)c(*)s3)c3nc4c(nc23)C(=C(C#N)C#N)c2ccccc2-4)cc1CC] | −4.60 | −3.60 | 1.00 | 1237 |
| Comparative Model 2-5 | 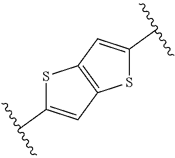 [SMILES: *c1cc2sc(*)cc2s1] | 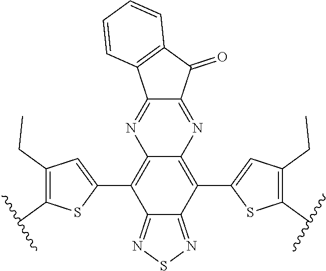 [SMILES: *c1sc(-c2c3nsnc3c(-c3cc(CC)c(*)s3)c3nc4c(nc23)C(=O)c2ccccc2-4)cc1CC] | −4.62 | −3.46 | 1.16 | 1065 |
| Comparative Model 2-6 | 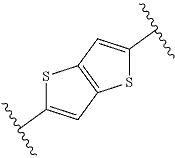 [SMILES: *c1cc2sc(*)cc2s1] | 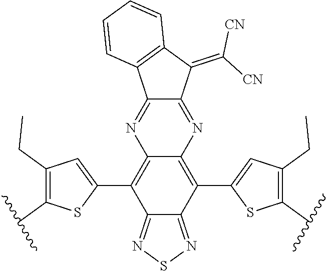 [SMILES: *c1sc(-c2c3nsnc3c(-c3cc(CC)c(*)s3)c3nc4c(nc23)C(=C(C#N)C#N)c2ccccc2-4)cc1CC] | −4.80 | −3.71 | 1.09 | 1140 |
| Comparative Model 2-7 | 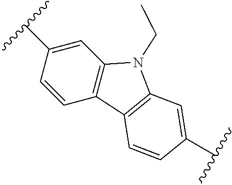 [SMILES: *c1ccc2c3ccc(*)cc3n(CC)c2c1] | 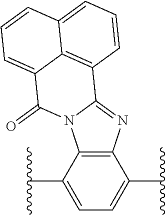 [SMILES: *c1ccc(*)c2c1nc1c3cccc4cccc(c(=O)n12)c43] | −5.00 | −2.43 | 2.58 | 481 |
| Comparative Model 2-8 | 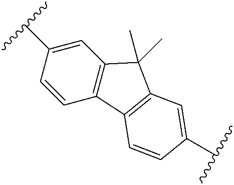 [SMILES: *c1ccc2c(c1)C(C)(C)c1cc(*)ccc1-2] | 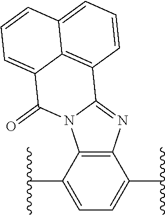 [SMILES: *c1ccc(*)c2c1nc1c3cccc4cccc(c(=O)n12)c43] | −5.06 | −2.43 | 2.63 | 472 |
| Model Example 2-5 | 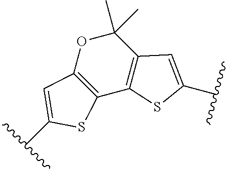 [SMILES: *c1cc2c(s1)-c1sc(*)cc1C(C)(C)O2] | 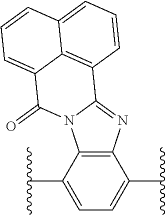 [SMILES: *c1ccc(*)c2c1nc1c3cccc4cccc(c(=O)n12)c43] | −4.46 | −2.53 | 1.93 | 643 |
| Model Example 2-6 | 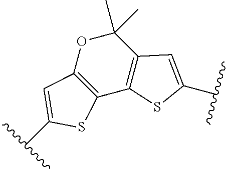 [SMILES: *c1cc2c(s1)-c1sc(*)cc1C(C)(C)O2] | 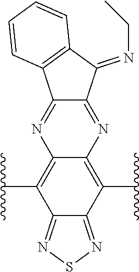 [SMILES: *c1c2nsnc2c(*)c2nc3c(nc12)/C(=N/CC)c1ccccc1-3] | −4.25 | −3.27 | 0.984 | 1260 |
| Model Example 2-7 | 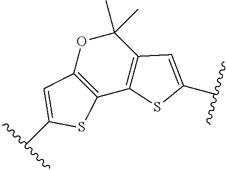 [SMILES: *c1cc2c(s1)-c1sc(*)cc1C(C)(C)O2] | 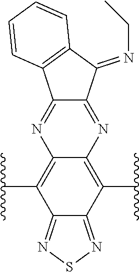 [SMILES: *c1c2nsnc2c(*)c2nc3c(nc12)/C(=N/CC)c1ccccc1-3] | −4.42 | −3.37 | 1.046 | 1185 |

### Device Example 1

A device having the following structure was prepared:
Cathode/Donor:Acceptor layer/Anode

A glass substrate coated with a layer of indium-tin oxide (ITO) was treated with polyethyleneimine (PEIE) to modify the work function of the ITO.

A mixture of Polymer Example 1 (donor) and ITIC-4F (acceptor) in a donor:acceptor mass ratio of 1:1.5 was deposited over the modified ITO layer by blade coating from a 15 mg/ml solution in 1,2,4 Trimethylbenzene; 1,2-Dimethoxybenzene 95:5 v/v solvent mixture. The film was dried at 80° C. to form a ca. 500 nm thick bulk heterojunction layer

An anode stack of MoO₃ (10 nm) and ITO (50 nm) was formed over the bulk heterojunction by thermal evaporation (MoO₃) and sputtering (ITO)

Device Examples 2-4 were prepared as described for Device Example 1 except that Polymer Examples 3-5 respectively were used in place of Polymer Example 1.

### Comparative Device 1

A device was prepared as described for Device Example 1 except that Comparative Polymer 1 was used in place of Polymer Example 1 and the ITO layer was 150 nm.

With reference to FIG. 2, dark current decreases in the order Device Example 1<Device Example 2<Device Example 3.

External quantum efficiency of Comparative Device 1 and Device Examples 1, 2 and 3 were measured. With reference to FIG. 4, Device Examples 1 and 3 produce a detectable signal at above >1300 nm whereas Comparative Device 1 does not.

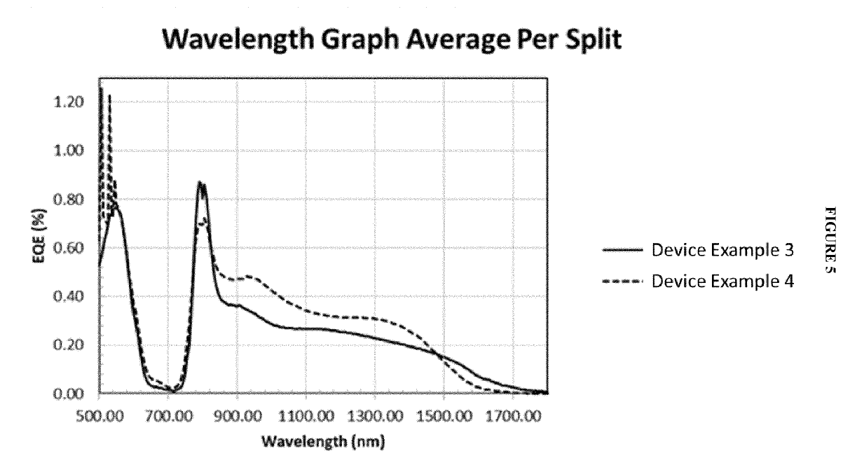

With reference to FIG. 5, Device Examples 3 and 4 both produce a signal in the range of about 750-1500 nm, with Device Example 4 giving higher efficiency in the range of about 800-1400 nm.

### Device Example 2

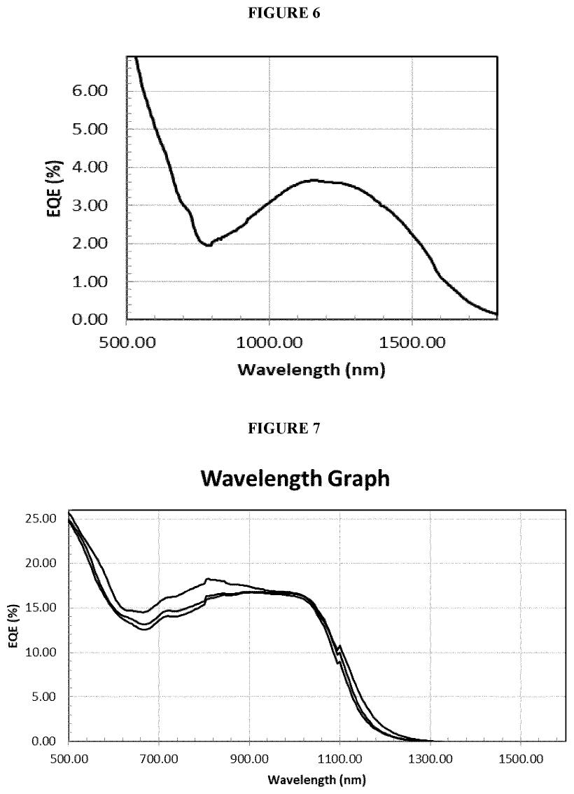

A device was prepared as described in Example 1 except that the bulk heterojunction layer was formed by depositing a blend of Polymer Example 6:fullerene KLOC-6 (1:1.75 by weight). As shown in FIG. 6, external quantum efficiency exceeded 3% at wavelengths in the range of about 1000-1400 nm.

### Comparative Device 2

A device was prepared as described in Device Example 2 except that Polymer Example 6 was replaced with Comparative Polymer 1.

With reference to FIG. 7, external quantum efficiency of Comparative Device 2 is lower than that of Device Example 2 at wavelengths above about 1200 nm.

1. A material comprising an electron-accepting unit of formula (I): 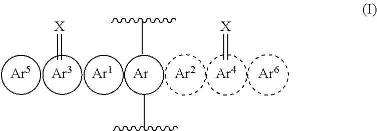
wherein:
Ar is a substituted or unsubstituted benzene or 6-membered heteroaromatic ring containing N and C ring atoms;
Ar¹ is a substituted or unsubstituted 5- or 6-membered heteroaromatic ring containing N and C ring atoms;
Ar² is a substituted or unsubstituted 5- or 6-membered heteroaromatic ring or is absent;
Ar³ is a 5-membered ring or a substituted or unsubstituted 6-membered ring;
Ar⁴ is a 5-membered ring or a substituted or unsubstituted 6-membered ring or is absent;
Ar⁵ is a substituted or unsubstituted monocyclic or polycyclic group containing at least one aromatic or heteroaromatic ring;
Ar⁶ is a substituted or unsubstituted monocyclic or polycyclic group containing at least one aromatic or heteroaromatic ring or is absent; and each
X is independently a substituent bound to a carbon atom of Ar³ and, where present, Ar⁴ with the proviso that at least one X is an electron-withdrawing group and wherein the material further comprises a conjugated electron-donating unit D of formula (II): 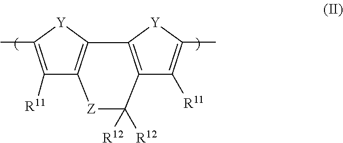
wherein:
Y in each occurrence is independently O, S or Se;
Z is O, S, C═O or NR³ wherein R³ is H or a substituent;
R¹¹ in each occurrence is independently H or a substituent; and
R¹² in each occurrence is independently a substituent.

2. A material according to claim 1 wherein the electron-accepting unit of formula (I) is selected from formulae (I-1) to (1-31): 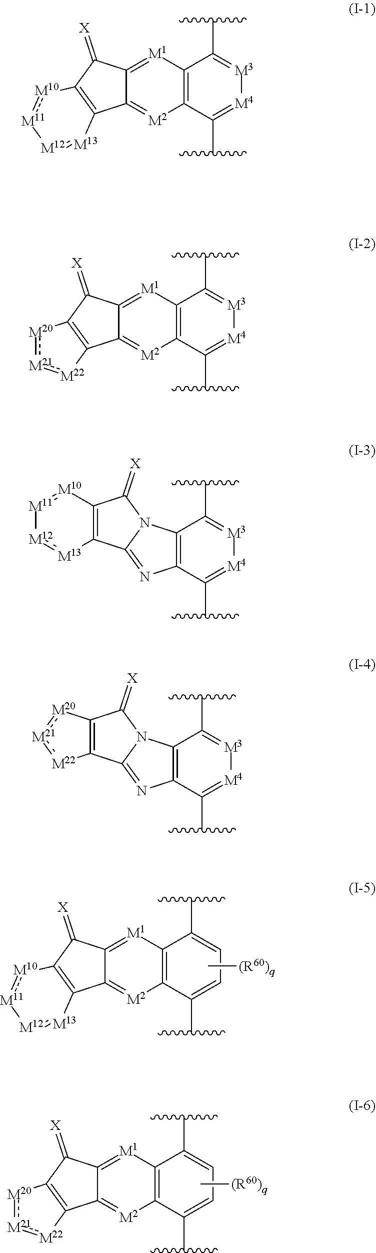 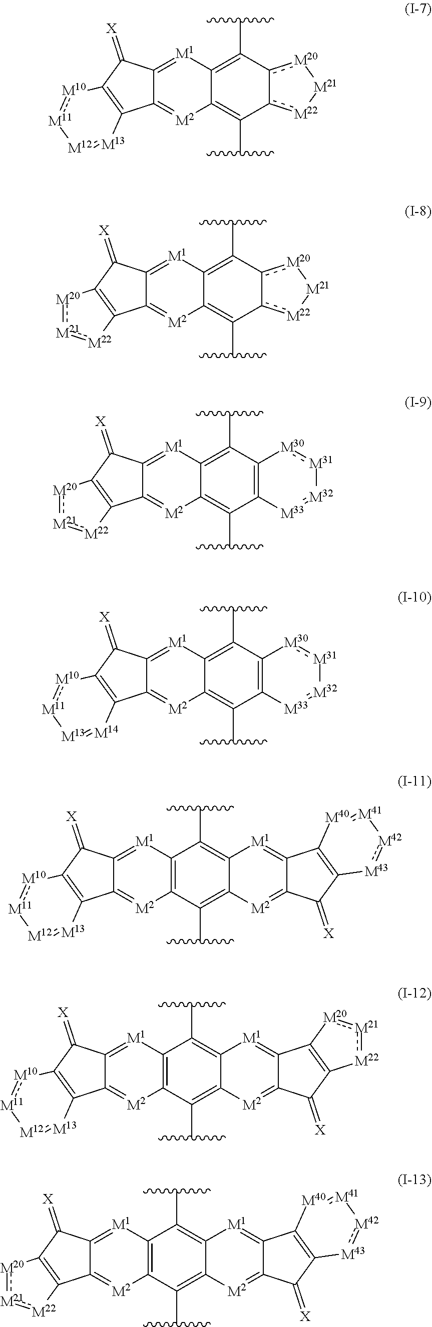 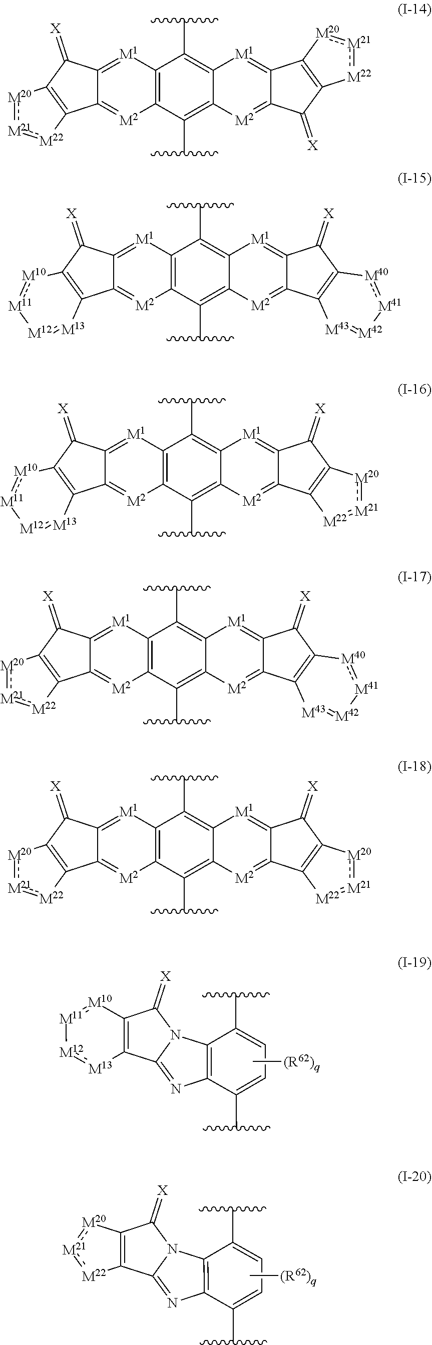 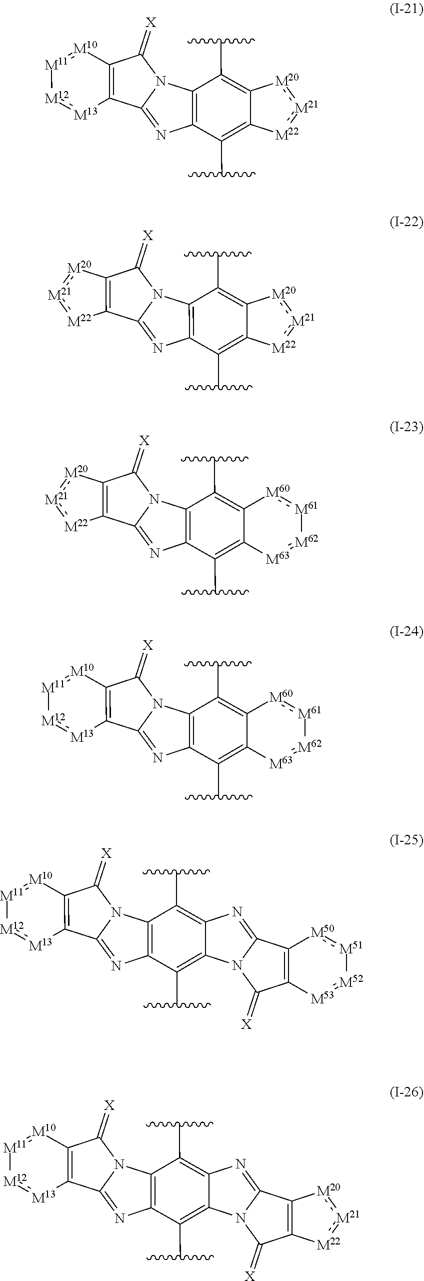 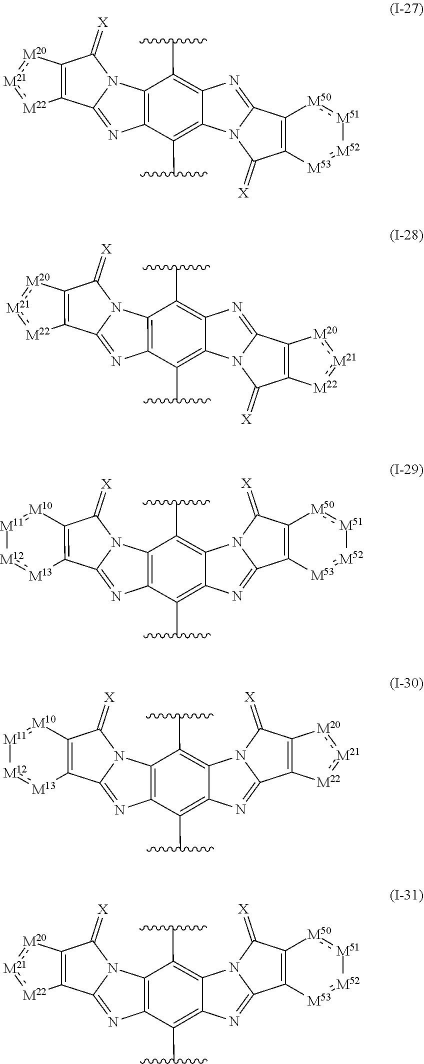
wherein
M¹, M², M³ and M⁴, is independently CR⁶¹ or N wherein R⁶¹ in each occurrence is a H or a substituent;
R⁶⁰ and R⁶² in in each occurrence is a H or a substituent;
M¹⁰, M¹¹, M¹², M¹³, M²⁰, M²¹, M²², M³⁰, M³¹, M³², M³³, M⁴⁰, M⁴¹, M⁴², M⁴³, M⁵⁰, M⁵¹, M⁵², and M⁵³ is independently N, S, O or CR⁶¹, wherein R⁶¹ in each occurrence is a H or a substituent and with the proviso that a S or O cannot be is not adjacent to another S or O;
X is independently an electron-withdrawing group; and
q is 0, 1, 2, 3 or 4.

3. A material according to claim 1 wherein the or each electron-withdrawing group X is independently selected from O, S and NX⁷⁰ wherein X⁷⁰ is selected from CN; COOR⁸⁰; C₁ to C₂₀ alkyl where one or more non-adjacent, not terminal C may be replaced by O or S; and a substituted or unsubstituted 5- or 6-membered aromatic or heteroaromatic ring; and CX¹⁰X¹¹ wherein X¹⁰ and X¹¹ are each independently F, Cl, Br, CN, CF₃ or COOR⁸⁰, wherein R⁸⁰ is a H or substituent.

4. The material according to claim 1 wherein the material is a non-polymeric compound.

5. The material according to claim 4 wherein the material is selected from formula (Ia)-(Id): 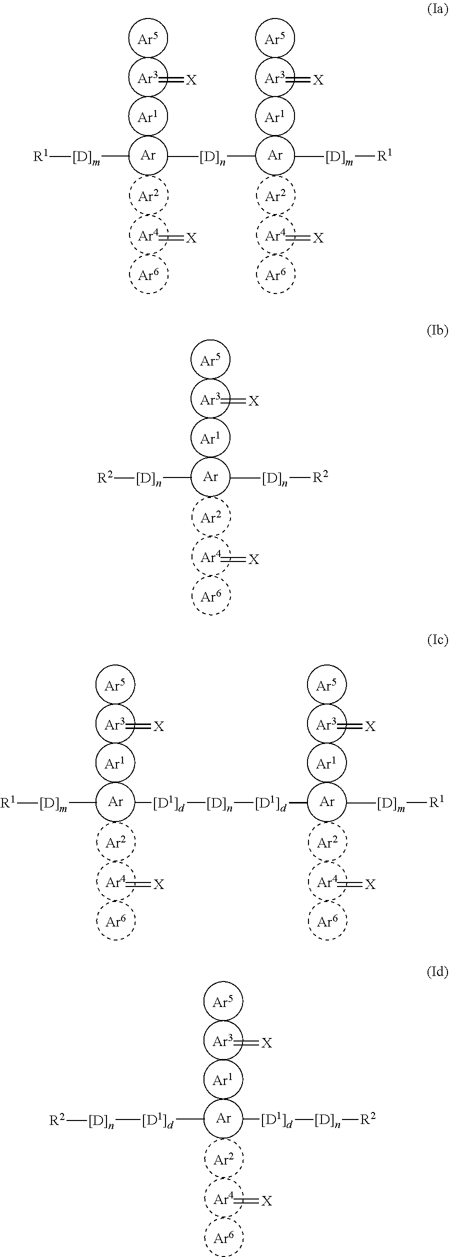
wherein n is at least 1; m is 0, 1, 2 or 3; d is 0, 1 or 2; D in each occurrence is independently a conjugated electron-donating unit D of formula (II); R¹ and R² independently in each occurrence is H or a substituent; and D¹ is a conjugated bridge unit, which is different to D.

6. The material according to claim 1 wherein the material is a polymer; the unit of formula (I) is an electron-accepting repeat unit of formula (I); and the electron-donating unit D is a conjugated electron-donating repeat unit of formula (II).

7. The material according to claim 6 wherein the polymer further comprises an electron-donating repeat structure D¹ which is different from conjugated electron-donating repeat unit D.

8. The material according to claim 6 wherein the polymer comprises a repeating structure of formula (Va): 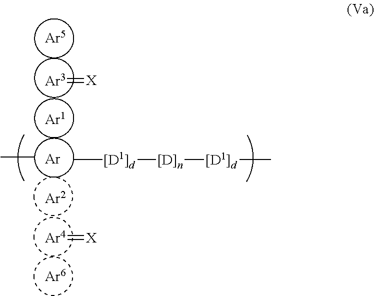
wherein d is 0, 1 or 2; D in each occurrence is independently a conjugated electron-donating unit D of formula (II); and D¹ is a conjugated electron donating bridge unit.

9. A composition comprising an electron donor and an electron acceptor wherein at least one of the electron donor and electron acceptor is a material according to claim 1.

10. The composition according to claim 9 wherein the electron acceptor is the material comprising an electron-accepting unit of formula (I).

11. The composition comprising an electron donor and an electron acceptor wherein at least one of the electron donor and electron acceptor is a material comprising an electron-accepting unit of formula (I) wherein the electron acceptor is the non-polymeric compound according to claim 5.

12. The composition comprising an electron donor and an electron acceptor wherein at least one of the electron donor and electron acceptor is a material comprising an electron-accepting unit of formula (I) wherein the electron donor is the polymer according to claim 6.

13. An organic electronic device comprising an active layer comprising a material or composition according to claim 1.

14. An organic electronic device according to claim 13 wherein the organic electronic device is an organic photoresponsive device comprising a bulk heterojunction layer disposed between an anode and a cathode and wherein the bulk heterojunction layer comprises a composition comprising an electron donor and an electron acceptor wherein at least one of the electron donor and electron acceptor is a material comprising an electron-accepting unit of formula (I).

15. An organic electronic device according to claim 14 wherein the organic photoresponsive device is an organic photodetector.

16. A photosensor comprising a light source and an organic photodetector according to claim 15, wherein the photosensor is configured to detect light emitted from a light source.

17. A photosensor according to claim 16, wherein the light source emits light having a peak wavelength of >1250 nm.

18. A formulation comprising a material or composition according to claim 1 dissolved or dispersed in one or more solvents.

19. A method of forming an organic electronic device according to claim 13 wherein formation of the active layer comprises deposition of a formulation comprising a material or composition comprising an electron-accepting unit of formula (I) dissolved or dispersed in one or more solvents onto a surface and evaporation of the one or more solvents.
